(19) United States
(12) Patent Application Publication
(10) Pub. No.: US 2004/0038920 A1
(43) Pub. Date: Feb. 26, 2004

(54) Chlamydial peptides and their mimics in demyelinating disease

(76) Inventors: Robert H. Swanborg, Clinton Township, MI (US); Derel C. Lenz, Farmington Hills, MI (US); Alan P. Hudson, Novi, MI (US); Judith A. Whittum-Hudson, Novi, MI (US)

Correspondence Address: VENABLE, BAETJER, HOWARD AND CIVILETTI, LLP, P.O. BOX 34385, WASHINGTON, DC, 20043-9998, US

(21) Appl. No.: 10/363,238

(22) PCT Filed: Sep. 6, 2001
(86) PCT No.: PCT/US01/27533

(51) Int. Cl. A61K 48/00; A61K 39/02

(52) U.S. Cl. 514/44; 424/190.1

(57) ABSTRACT

Subsequent to reports that Chlamydia pneumoniae (Cpn) was present in the CSF of a subset of multiple sclerosis (MS) patients, a 20-mer peptide from a protein specific to C. pneumoniae (Cpn) which shares a seven amino acid motif with a critical epitope of myelin basic protein (MBP), a major central nervous system antigen targeted by the autoimmune response in MS was identified. This bacterial peptide induces a Th1 response accompanied by severe clinical and histological experimental autoimmune encephalomyelitis in Lewis rats, a condition closely reflective of many aspects of MS. Various non-encephalitogenic peptide analogues and derivatives are disclosed and are useful for inhibiting such Th1 responses, inducing protective Th2 responses, and for treating a subject having MS or delaying onset of preventing MS in a subject at risk.

### BACKGROUND OF THE INVENTION

1. Field of the Invention

The present invention in the field of immunology and medicine is directed to a novel 20-mer peptide from Chlamydia pneumoniae that elicits autoimmune disease in an animal model of multiple sclerosis and has applicability in the diagnosis, prognosis and therapy of related demyelinating and neurodegenerative diseases.

2. Description of the Background Art

Multiple sclerosis (MS) is characterized by the presence of autoreactive T cells which target antigens associated with central nervous system (CNS) myelin, including myelin basic protein (MBP), proteolipid protein, and myelin oligodendrocyte glycoprotein (K. Ota et al., Nature 346:183 (1990); R. Martin et al., J. Exp. Med. 173:19 (1991); J. L. Trotter et al., J. Neuroimmunol. 33, 55 (1991); C. C. A. Bernard et al., J. Mol. Med. 75:77 (1997)). Detailed study of tissue samples from MS patients reveals demyelination and mononuclear cell infiltration of CNS white matter, oligoclonal immunoglobulin in the cerebrospinal fluid (CSF), and in many cases axonal degeneration (U. Traugott et al., Science 219, 308 (1983); Raine, C. S. et al., Ann. Neurol. 46:144 (1999); B. D. Trapp et al., N. Engl. J. Med. 338:278 (1998)). Patients with MS usually display one of two courses of disease progression, i.e., chronic disease of increasing severity or, more commonly, a remitting/relapsing disease form that progresses to incapacity at a slower rate. Because of its many clinical and immunopathologic similarities to MS, experimental autoimmune encephalomyelitis (EAE) in rodents has become a widely accepted model for study of the human disease. In the genetically susceptible Lewis (LEW) rat, immunization with a specific peptide from MBP (see below) induces an acute episode of paralysis that is mediated by infiltration of activated CD4+ inflammatory T cells into the CNS, thereby duplicating this and other important characteristics of MS pathology (S. S. Zamvil et al., Annu. Rev. Immunol. 8:579 (1992); R. H. Swanborg, Clin. Immunol. Immunopathol. 77:4 (1995)).

The etiology of MS remains elusive, but one explanation put forth for disease development postulates that specific antigenic epitopes from an unspecified infectious agent or agents induce(s) a host immune response in which cross-reactivity with myelin triggers disease, a concept referred to as molecular mimicry (R. S. Fujinami et al., Science 230, 1043 (1985)). In this scenario, certain T cells and/or antibodies produced in response to antigens from the infectious agent also recognize relevant self-antigens in the CNS, thereby initiating the destructive autoimmune process. To date little direct evidence exists to support the molecular mimicry hypothesis, although some data appear to support an infectious cause for MS (S. S. Soldan et al., Nature Med. 3, 1394-1397 (1997) and see below). Further, studies in mice have shown that infection with Theiler's virus elicits an inflammatory response in the CNS which does progress to relapsing/remitting EAE (Y. Katz-Levy et al., J. Clin. Invest. 104, 599 (1999)).

Interestingly, recent studies have suggested that Chlamydia pneumoniae, an unusual respiratory pathogen, is present in the CSF of a subgroup of MS patients but not in that of control subjects, in turn suggesting that infection with this obligate intracellular bacterium might be a triggering event in MS (S. Sriram et al., Ann. Neurol. 46, 6 (1999); G. Layh-Schmitt et al., Ann. Neurol. 47, 652 (2000)). The mere presence of C. pneumoniae in the CSF does not prove that the organism initiates the pathogenesis ending in MS. Rather, chlamydial infection of the CNS simply could be an opportunistic, secondary event in the disease; even in this circumstance, however, presence of the organism may exacerbate a pathogenic process initiated by other mechanisms.

The present inventors have employed the well-characterized LEW rat model of MS to investigate a causal relationship between infection with C. pneumoniae and MS.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present inventors have discovered that the peptide RFPNHYGCLLPRNPRTEDQN (SEQ ID NO: 3), corresponding to a C-terminal fragment of the C. pneumoniae gene and protein designated Cpn0483 and having homology to rat and guinea pig (“gp”) MBP is capable of inducing MS-like symptoms of EAE in rats. This disease is associated with specific reactivity of T lymphocytes to these peptides in vitro. These results support a causal mechanism involving molecular mimicry in human MS.

The present invention is directed to compositions, including substitution, addition and deletion variants and chemical derivatives of the Cpn0483 peptide (SEQ ID NO: 3), and peptidomimetics. These compositions are used to protect a subject, prophylactically, from development of MS or to treat various symptoms and immunopathological manifestations of MS to alleviate symptoms or to curtail disease progression.

An additional embodiment provides approaches to inducing protective T cells, preferably Th2 cells, capable of preventing, suppressing, or treating MS or an MS-like autoimmune disease. Such T cells may be prepared by (a) removing T cells from a subject susceptible to the disease, (b)expanding the T cells of step (a) in culture in the presence of the peptides of the present invention,; and (c) preparing protective T cell from the expanded cultured T cells. Such T cells, preferably obtained from the individual being treated, are then administered to the individual to prevent, suppress or treat the disease.

Specifically, the present invention provides composition comprising a Chlamydia pneumoniae (Cpn) protein 0483 (SEQ ID NO: 2) or a peptide fragment thereof or functional derivative of the peptide fragment, that stimulates Th1 cells that are specific for a myelin basic protein (MBP) autoantigen and, which, when administered to Lewis rats, induces severe experimental autoimmune encephalomyelitis in a majority of animals. The composition may be one encoded by all or part of the DNA molecule having SEQ ID NO: 1.

In one embodiment of the composition, the peptide includes the motif YGxLxxxxxRTxDxN (SEQ ID NO: 17), wherein x is any amino acid.

The above peptide fragment may have the sequence SEQ ID NO: 3.

In the above composition, a preferred peptide is RFPNHYGCLLPRNPRTEDQN (SEQ ID NO: 3) or is a Th1-stimulatory and/or encephalitogenic functional derivative thereof, for example RFPNHYGCLLPRNPRTEAQN (SEQ ID NO: 4) or RFPNHYGSLLPRNPRTEDQN (SEQ ID NO: 11).

The present invention also provides a compositions that is a non-encephalitogenic polypeptide comprising SEQ ID NO: 3, or that comprises a peptide analogue or functional derivative of SEQ ID NO: 3.

Preferred peptide analogues are:

| | | |
|---|---|---|
| (a) | RFPNHYGCLLPRNPATEDQN; | (SEQ ID NO:5) |
| (b) | RFPNHYGCLLPRNPNTEDQN; | (SEQ ID NO:6) |
| (c) | RFPNHYGCLLPRNPETEDQN; | (SEQ ID NO:7) |
| (d) | RFPNHYGCLLPRNPRTEDQA; | (SEQ ID NO:8) |
| (e) | RFPNHYGCLLPRNTPRTEDQR; | (SEQ ID NO:9) |
| (f) | RFPNHYGCLLPRNPRTEDQD; | (SEQ ID NO:1O) |
| (g) | YGCLLPRNPRTEDQN; | (SEQ ID NO:12) |
| (h) | PNHYGCLLPRNPRTEDQN; | (SEQ ID NO:13) |
| (i) | RFPNHYGCLLPRNIPR; | (SEQ ID NO:14) |
| (j) | NHYGCLLPRNPRTED; and | (SEQ ID NO:15) |
| (k) | HYGCLLPRNPRTED. | (SEQ ID NO:16) |

Also provided is a conjugate consisting of the above polypeptide, peptide or functional derivative conjugated to a second molecule, such as a detectable label, another polypeptide, or a small organic molecule.

This invention is also directed to a complex between and MHC class II protein and a peptide which complex is capable of inducing unresponsiveness or less responsiveness in a T cell that is specifically immunoreactive with an autoantigen and which T cell is an effector cell or regulatory cell in the pathogenesis of MS, the complex comprising

(a) a peptide that includes SEQ ID NO: 3 or includes a functional derivative thereof covalently bound to

(b) an isolated MHC class II component having an antigen binding pocket,

wherein the antigenic peptide is physically associated with the antigen binding pocket and is recognized by the T cell receptor of the reactive T cell.

The peptide may be covalently bound via a peptide linkage to an MHC class II chain and non-covalently associated with the antigen binding pocket.

In the complex, the peptide preferably comprises an epitope recognized by a T cell receptor of a T cell specifically immunoreactive with an MBP autoantigen.

In a preferred complex, the peptide is a non-encephalitogenic peptide analogue or functional derivative of SEQ ID NO: 3.

The present invention provides pharmaceutical composition comprising any of the above compositions or complexes and a pharmaceutically acceptable carrier or excipient.

Also provided is a method for inhibiting a Th1 lymphocyte response to a peptide or protein that includes the peptide motif YGxLxxxxxRTxDxN (SEQ I) NO: 17), wherein x is any amino acid, or that is induced by Cpn0483 peptide SEQ ID NO: 3, which method comprises providing, preferably in vivo, to a population of lymphocytes that includes Th1 cells an effective amount of the non-encephalitogenic composition above or the complex comprising a non-encephalitogenic peptide above, to inhibit the Th1 response.

Another embodiment is a method for inducing a Th2 immune response to an autoantigenic peptide associated with MS in a subject, comprising contacting the immune system of the subject, preferably in vivo, with the non-encephalitogenic composition above or the complex comprising a non-encephalitogenic peptide above, optionally in combination with a cytokine, preferably IL-4, or other agent that promotes activation of Th2 lymphocytes.

Also provided is a method of treating a subject having, or at risk for, multiple sclerosis and in need of treatment or prophylaxis therefor, comprising administering to the subject an effective amount of the above pharmaceutical composition.

In another embodiment, the invention provides a genetically modified mammalian cell comprising a polynucleotide encoding a Cpn polypeptide 0483 (SEQ ID NO: 2), a Cpn peptide SEQ ID NO: 3, or a functional derivative of the peptide.

The genetically modified mammalian cell may comprise a polynucleotide having SEQ ID NO: 1 or a fragment thereof which polynucleotide or fragment is expressed in or on the cell, the polypeptide or peptide product of which stimulates Th1 cells that are specific for a MBP auto antigen.

Preferably, the genetically modified mammalian cell comprising a polynucleotide encoding a non-encephalitogenic (a) polypeptide comprising SEQ ID NO: 3, or (b) peptide analogue or functional derivative of SEQ ID NO: 3.

Also included is a population of cells that were transfected or transduced with an exogenous polynucleotide that encodes the peptide or functional derivative above, wherein the cells express the polypeptide, peptide or functional derivative when they are administered to a subject in vivo, and wherein, when the subject has multiple sclerosis, the presence of the polypeptide, peptide or functional derivative delays onset, prevents or diminishes the progression or severity or the multiple sclerosis.

Preferred cells, above, are human cells.

Another method of treating a subject having, or at risk for, multiple sclerosis and in need of treatment or prophylaxis therefor, comprises administering to the subject an effective amount of cells above that express a non-encephalitogenic protein or peptide, and that are autologous or otherwise compatible, e.g., histocompatible, with the subject, thereby treating the subject.

Also provided is a method of treating a subject having, or at risk for, multiple sclerosis and in need of treatment or prophylaxis therefor, comprising the steps of:

(a) obtaining T cells from the subject,

(b) optionally, enriching Th2 cells from the T cells;

(d) expanding T cells of step (a) or (b) in culture in the presence of the non-encephalitogenic composition above, optionally in the presence of growth factors or accessory or feeder cells; to obtain protective T cells; and

(e) administering to the subject an effective amount of the protective T cells, thereby providing the treatment or prophylaxis to the subject.

The above method may include administering to the subject (a) an agent with promotes the survival or action of the cells, and/or (b) a drug that treats any symptom of multiple sclerosis.

### BRIEF DESCRIPTION OF THE DRAWINGS

FIG. 1 shows an RT-PCR analyses demonstrating expression of C. pneumoniae coding sequence Cpn0483 in infected Hep-2 cells in vitro. Lanes are: 1, negative (water) control; 2, positive PCR control using purified C. pneumoniae DNA as template; 3, negative control using DNA from uninfected Hep-2 cells as template; 4, negative control using RNA from C. pneumoniae elementary bodies as template; 5-7, analysis of cDNA made from RNA obtained from Hep-2 cells infected for 24, 48, and 72 hr, respectively; 8, analysis of RNA from the 48 hr infected cells in the absence of reverse transcription.

FIG. 2 shows the clinical course of EAE. LEW rats were immunized with 50 μg Cpn0483 (●) or MBP68-86 (▴). Vertical axis: mean clinical score (maximal grade=3.0); horizontal axis: day post-immunization.

FIGS. 3 and 4 show results measuring T cell proliferative responses. Cpn0483-primed T cells respond to priming peptide and cross-react with ratMBP68-86 (FIG. 3); and ratMBP68-86-primed T cells respond to priming peptide and cross-react with chlamydial peptide (FIG. 4).

### DESCRIPTION OF THE PREFERRED EMBODIMENTS

The amino acid sequence of the C. pneumoniae encoded protein designated Cpn0483 (SEQ ID NO: 2) has the sequence shown below. The peptide 20-mer near the C-terminus (SEQ ID NO: 3) shares homology with rat and guinea pig MBP and is underscored below. The biological function of this Cpn protein is currently unknown.

| | |
|---|---|
| LIKKRAIFER MFPIPPPHCP PNNKNNFYHL TTDTKDPLLL RILRTIGYVL | 50 |
| LHIITLGLLL LIHYYKHHRV VRKEGLPTPP TLPKGPEPKT IEIAKQPPKD | 100 |
| GEDKKPDVPK PGTPPPEDTP PPPPKAPSPA SPKVPKQPAD KKPTPPREAP | |
| PPPVRVATPM PLRPSSQGYW QCLNRMVSMV LRRAPLPLPA MQVDPILGDF | 200 |
| NPHFVASYPN RIDNEPMYFQ IKQFKKIAQN PDLPQQHRRL AQLSLEQALY | |
| LNDNYYLVNV PGDGNCFYRA YAVGWLSALY EESSRNDIVF EQEATRLLDL | 300 |
| PFASSSPANA NLCAEMAELL QLCSTYCSFI DLYDGVILSQ KHTATLIAFL | |
| RKLSAYAIRQ QIAASSNEET ARALFISDMQ DDLLPSVLEF LAANRPYSEL | 400 |
| FQNLIDHSAL PYMQSRDKLF LLLEHLPALF LTDAELQKMS PEDQQLRKQY | |
| EREIREAFAK LSRRIADSGW DTERFNAIVK DHLPEAIRCQ YSRFLATIEN | 500 |
| RRSGDLPWSP ALSFFAFLCT CPSVRFHKLC ATFYKSLEDI IIASAPPQRS | |
| IQEILQISNA SLSYLNEDLD SSWQREVISS NIMTILTTHE SLTLESSMPQ | 600 |
| LETLHKRIAN LLKNVISTSF ETPPLSNQPD LLSNLVNKLL VAIHSKLELK | |
| EHFNTVCSAR SLRLTRDEGS GLSQEQDLLY TQAVQLLFFI LQHPQVNNRP | 700 |
| ETKDAVKELK MLLLPFLQYA FKKVENEKKL QKLLRSILGS LVLKPPARYP | |
| STPSNKDKET FCKFWSRHPE VMVLDPILEK NCMQFLRATF PNYQLETEAI | 800 |
| LLEKEIESTF RNGWNVFLTR LNLFGSKLGS PSSPTALSDQ FSKSFLIFCF | |
| LNNYPKLLQK KTPLAARLDA FQREASHRFT QVKDKLLLSL KYGFPLATAT | 900 |
| INQYSRARDQ LICNLLKNTV TASDGFCRSG FRQSLIGYLH SLSSNELGDI | |
| LDDVKEQAEA NDVAAMTTVP LQPFAVCLIM SDRDTVSEEN IENFVAMHGF | 1000 |
| LNTISPERDA RIFLIRFPNH YGCLLPRNPR TEDQNSKPDS SNP 1043 | |

This putative gene is present in C. pneumoniae but not in other species of Chlamydiae. The DNA coding sequence is SEQ ID NO: 1, above. This DNA encodes a 1043-amino acid protein (SEQ ID NO: 2) of unknown function.

The C. pneumoniae DNA sequence Cpn0483 (SEQ ID NO: 1) that encodes the protein SEQ ID NO: 2 is:

TTG ATT AAA AAA CGA GCA ATT TTT GAA CGT ATG TTT CCA ATT CCC CCA CCA CAT TGC
CCG CCC AAT AAC AAG AAT AAT TTT TAC CAC TTA ACG ACT GAT ACT AAA GAC CCT CTG
TTA CTT AGA ATT CTA CGT ACC ATA GGA TAC GTT CTG CTC CAT ATC ATT ACT CTT GGT
TTG CTT CTT CTG ATT CAC TAC TAC AAG CAT CAT CGG GTT GTC AGA AAA GAA GGC TTG
CCA ACG CCT CCC ACT CTT CCC AAA GGA CCA GAG CCA AAA ACT ATA GAA ATT GCC AAA
CAA CCG CCT AAG GAT GGT GAA GAC AAA AAA CCC GAT GTT CCC AAG CCG GGC ACG CCG
CCC CCA GAG GAC ACA CCC CCG CCT CCC CCC AAA GCT CCT TCA CCA GCG AGC CCA AAA
GTC CCT AAA CAA CCT GCT GAT AAA AAG CCG ACT CCA CCA CCA GAG GCC CCT CCT CCT
CCC GTA CGG GTG GCT ACC CCC ATG CCT CTC CGC CCA TCT AGT CAA GGC TAT TGG CAA
TGC TTA AAT CGC ATG GTG AGC ATG GTA CTA AGA CGA GCG CCT CTG CCT CTT CCT GCC
ATG CAA GTT GAT CCA ATA CTT GGC GAC TTT AAC CCT CAT TTC GTA GCT TCC TAT CCC
AAT CGG ATT GAT AAC GAA CCG ATG TAT TTC CAA ATA AAA CAG TTC AAG AAA ATC GCA
CAA AAT CCG GAT CTT CCT CAA CAA CAC CGG CGA CTT GCG CAA CTC TCT CTT GAA CAG
GCT CTC TAT CTA AAT GAC AAT TAC TAC CTT GTG AAT GTA CCG GGA GAT GGG AAC TGC
TTT TAT CGT GCC TAT GCT GTA GGA TGG CTA TCT GCT CTC TAC GAA GAG AGC AGC AGA
AAT GAT ATT GTC TTT GAG CAG GAA GCC ACA CGT CTC CTT GAC CTG CCT TTC GCC TCC
TCT TCT CCG GCA AAT GCG AAT CTT TGT GCA GAA ATG GCT GAA CTC CTT CAG TTA TGC
AGT ACT TAT TGC TCC TTC ATA GAC CTC TAT GAC GGG GTG ATT CTT TCT CAG AAA CAC
ACT GCA ACT CTG ATA GCC TTT CTA AGA AAA CTC TCT GCA TAT GCG ATT CGC CAA CAA
ATC GCA GCT TCA AGT AAT GAA GAA ACA GCG AGA GCC TTA TTT ATT TCT GAT ATG GAG
GAC GAT CTC CTC CCC AGT GTT CTG GAA TTT CTT GCT GCA AAT CGT CCC TAT TCG GAA
TTG TTC CAA AAT CTC ATT GAT CAT TCC GCA CTT CCT TAC ATG CAA TCT AGA GAC AAA
CTC TTT CTT CTC TTG GAA CAT CTG CCC GCT CTC TTT CTT ACT GAT GCA GAG CTT CAA
AAG ATG TCT CCA GAA GAT CAA CAA CTT CGA AAG CAA TAT GAA AGA GAA ATA CGA GAG
GCT TTT GCT AAG CTG AGT CGA CGC ATT GCT GAT TCA GGG TGG GAT ACT GAG AGA TTC
AAT GCT ATA GTC AAA GAT CAC CTC CCT GAA GCA ATC CGA TGT CAA TAC TCT CGC TTT
CTT GGA ACT ATA GAA AAC AGA CGA TCT GGG GAT CTC CCT TGG TCT CGA GCT CTT TCT
TTC TTT GCT TTT CTA TGT ACC TGC CCC TCT GTA AGA TTT GAC AAA CTC TGC GCT ACT
TTC TAC AAA TCA TTA GAG GAT ATC ATT ATA GCG TCC GCG CCC CCC CAA CGC TCT ATA
CAA GAG ATC TTA CAA ATA AGT AAC GCC TCC CTC AGC TAC CTT AAT GAA GAT TTA GAT
TCT TCT TGG CAA CGA GAG GTG ATT TCT TCT AAC ATC ATG ACT ATC CTT ACG ACT CAT
GAG AGT TTG ACG TTA GAG AGC TCT ATG CCT CAA CTC GAA AGA CTA CAT AAA CGC ATA
GGA AAC CTA TTA AAG AAT GTA ATA TCC ACA TCC TTT GAA ACC CCT CCT TTA AGC AAT
GAG CCG GAT TTA CTT TGA AAT CTT GTA AAC AAG CTA TTA GTC GCA ATT CAT AGT AAG
CTT GAA TTA AAA GAG CAC TTC AAT ACT GTC TGC TCG GGA AGA AGT TTA CGT TTA ACG
CGT GAT GAA GGC AGT GGT CTC TGA CAA GAG GAG GAC CTC CTC TAT ACA GAG GCA GTA
GAG CTC TTA TTC TTT ATT TTA CAG CAT CCT CAA GTG AAT AAT CGT CGA GAA ACT AAA
GAT GCC GTT AAA GAG TTA AAA ATG CTT CTA CTT CCT TTT CTA CAA TAT GCC TTT AAA
AAA GTA GAA AAC GAA AAG AAA CTC CAA AAA CTT CTA CGT TCC ATT CTA GGG TCT CTA
GTA CTC AAG CCT CGA GGA CGC TAT CCT TCA ACC CCT TCT AAT AAA GAT AAA GAG ACG
TTC TGC AAG TTC TGG TGA CGA CAT CCT GAA GTG ATG GTT TTA GAT CCC ATA CTT GAA
AAG AAC TGT ATG GAG TTT CTA CGA GCT ACT TTC CCA AAT TAT CAA CTG GAA ACC GAG
GCC ATA CTC TTA GAA AAA GAA ATC GAA AGT ACC TTT AGG AAT GGG TGG AAC GTT TTT
TTA AGA CGG TTA AAT CTC TTC GGA TGA AAA CTG GGT TCG CCT TCT TCT CCC AGA GCT
TTA AGT GAT GAG TTT TCG AAA TCT TTT TTA ATC TTT TGT TTC CTT AAC AAC TAC CCT
AAA CTT CTA CAA AAA AAG ACT CCG CTA GCT GCT CGA TTA GAC GCT TTC CAA AGA GAG
GCT TCT CAT AGA TTT AGA CAA GTA AAA GAT AAG CTT TTA CTT TCG TTA AAA TAC GGT
TTC CCT CTA GCT AGA GCG ACT ATA AAT CAA TAC TCT AGA GCT CGA GAT GAG TTG ATT
TGT AAT CTC TTA AAA AAC ACG GTC AGA GGA TCT GAT GGT TTC TGT CGC TCT GGT TTT
AGA CAA TGA CTG ATA GGC TAC CTC GAC TCC CTA AGT TCT AAT GAA CTC GGT GAT ATC
TTG GAT GAC GTC AAA GAG CAA GCT GAG GCT AAC GAC GTC GCT GCT ATG ACT ACT GTA
CCT TTG GAG CCG TTT GCT GTT TGT CTG ATC ATG TCT GAT CGA GAT ACT GTC TGA GAA
GAA AAT ATT GAA AAC TTT GTT GCG ATG CAT GGA TTT TTA AAT AGA ATT TCT CCG GAA
AGA GAC GCT CGT ATC TTC TTA ATC CGC TTC CCC AAC CAC TAC GGT TGT CTC TTG CCT
AGA AAC CCT AGA ACT GAA GAT GAG AAC TGA AAA CCG GAC AGC TGA AAT CCC TAG

In the LEW rat, the dominant epitope for EAE induction is a peptide comprised of amino acids 68-86 of the guinea pig (gp) MBP molecule (G. A. Hashim, Science 196, 1219 (1977); M. D. Mannie et al., Proc. Natl. Acad. Sci. USA 82, 5515 (1985); R. B. Smeltz et al., J. Immunol. 162, 829 (1999) ). However, EAE is autoimmune, since the disease can be induced with self (rat) whole MBP or rat MBP68-86 peptide (SEQ ID NO: 18), which differs from the guinea pig peptide (SEQ ID NO: 21) by a Thr-for-Ser substitution at position 80 (R. Weissert et al., J. Immunol. 160, 681 (1998)) (Table 1).

The present inventors sought to identify a peptide from some protein specific to C. pneumoniae that shows marked sequence similarity to rat MBP68-86 peptide. A computer-assisted search of the complete genome of the organism was performed using software and information provided by NCBI at the NIH website to search for potential peptides unique to C. pneumoniae having high homology with rat MBP68-86. A Blast search of the entire chlamydial genome identified a peptide encoded by Cpn0483 gene that was 71% homologous (counting identical residues+similar residues) to ratMBP68-86. This peptide (SEQ ID NO: 3) was near the C-terminus of the Cpn0483 full length protein (SEQ ID NO: 2), as shown above. See, also, Table 1

TABLE 1
Amino acid sequences of MBP68-86
and Cpn0483 peptides.

| | | |
|---|---|---|
| Rat 68-86 | YGSLPQKSQRTQDENPV | SEQ ID NO:18 |
| Gp 68-86 | YGSLPQKSQRSQDENPV | SEQ ID NO:21 |
| Cpn0483 | RFPNHYGCLLPRNPRTEDQN | SEQ ID NO:3 |

Suitable peptides or polypeptides to be used in the present invention preferably comprise SEQ ID NO: 3 or an analogue thereof. The peptides may be as short as 20 amino acids or even shorter if the truncation does not interfere with the peptide's desired biological activity. Preferred peptides 20-60 residues, more preferably 20-40, more preferably 20-30. The additional amino acid residues must be ones that do not inhibit, and preferably promote the biological activity, for example by enhancing stability in solution, biological half life, etc.

The Cpn peptides of the present invention are recognized by the immune system (either by T cells or antigen-presenting cells (APCs) and are capable of inducing the proliferative response of a subject's T lymphocytes to an MS autoantigen, particularly, MBP or an encephalitogenic or T cell stimulatory peptide thereof or inhibiting such induction. Antagonist peptides are those that bind to the TCR of the relevant T cells and either block its ability to bind other pathogenic autoantigenic polypeptides present in the cells' milieu or actively reverse the cells' activation by such autoantigens. The non-encephalitogenic Cpn peptide variants (analogues, truncated peptides) are considered candidate antagonists useful as protective or therapeutic agents for the prophylaxis or treatment of MS. These include the peptides SEQ ID NO: 5, 6, 7, 8, 9, 10, 12, 13, 14, 15 and 16.

In the case where the mechanism of action in inhibiting development or progression of MS is through the action of regulatory or “suppressor” T cells that act to limit the effector response. Agonist peptides that stimulate the activity of such regulatory T cells are preferred. The present Cpn peptides that are non-encephalitogenic (noted above) are candidate agonists of such regulatory T cell activity.

More preferable for therapeutic uses are those peptides which inhibit the stimulation of patient effector T lymphocytes by the autoantigen and thereby protect a subject from an immune-related neurodegenerative disease, such as MS. A Cpn peptide or analogue that include the T cell epitope, when administered to a subject in a therapeutic regimen, is capable of modifying the response of the individual to the autoantigen leading to inhibition or reversal of the autoimmune response. This permits either prevention or treatment of the disease state in a subject, including a human.

The present invention is directed to use of the peptide compositions to “protect” an individual from MS or an immune-related MS-like disease which includes other immune-mediated demyelinating and neurodegenerative diseases. The term “protect” or “protection” from the disease as used herein is intended ton encompass “prevention,” “suppression” or “treatment” of the disease. “Prevention” involves administration of the protective composition prior to the induction of the disease. Thus, for example, in the animal model, EAE, successful administration of a protective composition prior to injection of the encephalitogen that induces the disease results in “prevention” of the disease.

“Suppression” involves administration of the composition after the inductive event but prior to the clinical appearance of the disease. Again, using EAE as an example, successful administration of a protective composition after injection of the encephalitogen, but prior to the appearance of neurological symptoms comprises “suppression” of the disease.

“Treatment” involves administration of the protective composition after the appearance of the disease. In the EAE example, successful administration of a protective composition after injection of the encephalitogen and after clinical signs have developed comprises “treatment” of the disease.

It will be understood that in human medicine, it is not always possible to distinguish between “preventing” and “suppressing” since the ultimate inductive event or events may be unknown, latent, or the patient is not ascertained until well after the occurrence of the event or events. Therefore, it is common to use the term “prophylaxis” as distinct from “treatment” to encompass both “preventing” and “suppressing” as defined herein. The term “protection,” as used herein, is meant to include “prophylaxis.”

Identification of modified or substituted peptides (analogues) or peptidomimetics (such as the “functional derivatives” described below), collectively referred to here as “peptides,” which are useful in the diagnostic and therapeutic methods of the present invention can be easily accomplished by testing their ability to inhibit proliferative responses in vitro of patient T cells, or the Cpn0483 peptide-specific T cell lines or clones, or inhibit binding of the Cpn0483 peptide to antibodies. Any shorter immunodominant epitopes in the Cpn0483 peptide sequence which are targeted by antibodies and/or T cells in MS patients are identified by use of truncated and/or a series of peptides that “walk” the Cpn0483 peptide in five amino acid steps. These peptide epitopes are tested for their “antigenicity” in eliciting T cell proliferation or in binding to antibodies. Epitope specificity and affinity of the Cpn0483 peptide autoantibodies or TCRs specific for such epitopes is assessed in the appropriate body fluid.

According to the present invention, peptides are provided which can compete efficiently with RFPNHYGCLLPRNPRTEDQN (SEQ ID NO: 3) for recognition by T lymphocytes which are associated with MS or another immune-related disease which involves reactivity to the Cpn0483 peptide. In another embodiment, the peptides compete with RFPNHYGCLLPRNPRTEDQN (SEQ ID NO: 3) for recognition by a T cell line or clone specific for this peptide. Modification of residues critical for T cell activation will yield a T cell antagonist peptide that can specifically inhibit the proliferative response to “native” Cpn0483 peptide (see Examples).

By the term “the Cpn0483 peptide-specific T cell” is intended any T lymphocyte, T lymphocyte line or clone, which is immunoreactive with RFPNHYGCLLPRNPRTEDQN (SEQ ID NO: 3) or a fragment thereof. Immunoreactivity with the Cpn0483 peptide is intended to include binding of the Cpn0483 peptide (or fragment thereof) or the stimulation of the cells' biologic activity including protein synthesis, DNA synthesis, blastogenesis, cell proliferation, aggregation or cytotoxicity. Such T lymphocytes are particularly associated with MS or other neurodegenerative diseases characterized by the presence of the MBP reactivity. Production of the Cpn0483 peptide specific T cell lines and clones is described below.

The present invention is intended to include fragments and functional derivatives of the peptides which maintain, and preferably improve, their functional characteristics in vitro or in vivo (see below), including their pharmaceutical characteristics. Preferably a functional derivative is one which retains immune reactivity with anti-RFPNHYGCLLPRNPRTEDQN antibodies, T lymphocytes or both. Functional derivatives include a the Cpn0483 peptide having one or more amino acid substitutions. Such substitutions may render the peptide incapable of stimulating the Cpn0483 peptide-specific T cells but capably of inactivating or tolerizing such T cells, as is known in the art.

Amino acid substitutions in the Cpn0483 peptide are introduced during chemical synthesis. Creation of single amino acid substitutions in the Cpn0483 peptide allows testing of the role of each amino acid in the formation of three-dimensional epitopes. Recognition of native and altered peptides by antibodies is be performed by conventional enzyme immunoassay (EIA) or by T cell proliferation studies as described herein.

It is known that substitution of a single amino acid may significantly alter T-cell immunogenicity of a peptide. Moreover, a single amino acid substitution can also transform an antigenic peptide into a TCR antagonist peptide (Franco, A. et al., 1994, Eur. J. Immunol. 24:940-946). Since T cells recognize peptide fragments bound to MHC proteins, immunodominant T cell epitopes in the Cpn0483 peptide are also evaluated using synthetic peptides (see below). To define the role of specific amino acids in formation of B-cell versus T-cell epitopes, substitution variants of the Cpn0483 peptide are tested in antibody binding and T cell proliferation assays described herein.

Altered peptides generated by single amino acid substitution of the antigenic peptide can alter the patterns of differentiation and effector functions of the responding T lymphocytes Nicholson L B and Kuchroo V K, Crit Rev Immunol, 1997, 17:449-462. By defining the pattern of recognition and residues of the cognate ligand that bind to the TCR, altered peptide ligands (APLs) can be generated by selective substitution of TCR contact residues in the antigenic peptide. These APLs have been utilized in the art in studies in vitro to characterize T cell function. In vivo APLs have been utilized to study regulation of autoimmune diseases. Based on knowledge or which APLs that hypo- or hyper-stimulate T cell function, one can design specific peptides to alter (inhibit or enhance) immune responses in vivo in autoimmune disease. According to the present invention, the Cpn0483 peptide is substituted singly or doubly and tested in routine assays for encephalitogenicity and T cell reactivity in vitro or in vivo to determine which altered ligands of this peptide are useful in the treatment or prophylaxis of MS.

Analysis of peptide suggests that different subsets of T cells respond to MBP and to the Cpn0483 peptide or its substitution variants. This is shown in Example ______?. This is believed to be based on secondary contacts with the TCR of the responding T cell.

In the native Cpn0483 sequence (SEQ ID NO: 3) the R underlined below

RFPNHYGCLLPRNPRTEDQN

is believed to be a primary contact residue with the TCR because substitution of that R with either A or N abrogates encephalitogenic activity (see Example V). The C-terminal N residue is also important in activity, as its replacement with A also diminished encephalitogenicity. As is unusual for the C-terminal residue in an MHC-binding and TCR-binding peptide to be a primary TCR contact residue, it is believed that the latter effect is de to a change in folding caused by this C-terminal substitution which secondarily affects the primary contact residue such as the R indicated above.

Regulatory T cells such as Th1, Th2 (T suppressor cells) may also play a role in mediating the protective effects in EAE and MS. It is generally accepted in the art that a Th2 response is desirable to prevent or overcome a Th1 response or effector T cell response that leads to immunopathology. In animal model such as rat EAE, routine in vivo cell transfer studies permits determination of which cell type is protective or pathogenic in a given setting.

Unlike B cells which recognize three-dimensional epitopes often comprised of discontiguous amino acids, T cells bind to linear epitopes. A panel of synthetic peptides overlapping by any selected number of amino acids, may be used to map antigenic epitopes driving the Cpn0483 peptide-specific immune responses in MS. Overlapping synthetic peptides have been successfully used to analyze T cells responses to myelin antigens, including MBP and PLP.

MS patient sera are tested in EIA with the Cpn0483 peptide to detect recognition of conformational epitopes that may have escaped detection by Western blot. Recognition of individual the Cpn0483 peptide epitopes are revealed with a panel of overlapping synthetic peptides as antigens. Human MBP or an peptide that includes MBP69-86 can serve as a reference antigen. A known anti-Cpn0483 peptide polyclonal antibody or mAb serves as a positive control. Epitopes identified in this way are further analyzed using known methods.

The above peptides in combination with the T cells, antibodies and immunoassays described herein will permit rapid and repeated evaluation of disease-associated changes in the titer and epitope specificity of any anti-Cpn0483 peptide antibodies in serum and CSF. These compositions and methods are also useful to monitor changes occurring during the course of therapy.

The term “functional derivative” of the Cpn0483 peptide as used herein refers to a molecule substantially identical to this peptide in which one or more amino acid residues have been replaced (substitution variant) or which has one or several residues deleted (deletion variant) or added (addition variant). A “fragment” of the peptide refers to any subset of the molecule, that is, a shorter peptide. The term “functional derivative” as used herein also means a “chemical derivative” or peptidomimetic.

A functional derivative of the peptide of the invention, which retains at least a portion of the function of the peptide which permits its utility in accordance with the present invention, that is, binding to anti-the Cpn0483 peptide antibody, induction of anti-the Cpn0483 peptide antibody, and binding to and/or reaction with T lymphocytes specific for the Cpn0483 peptide or for MBP or binding to a soluble TCR from such T cell particularly when the peptide is complexed with an MHC class II molecule.

A “variant” of the Cpn0483 peptide refers to a molecule substantially identical to this peptide in which one or more amino acid residues have been replaced (substitution variant) or which has one or several residues deleted (deletion variant) or added (addition variant). A “fragment” of the peptide refers to any subset of the molecule, that is, a shorter peptide.

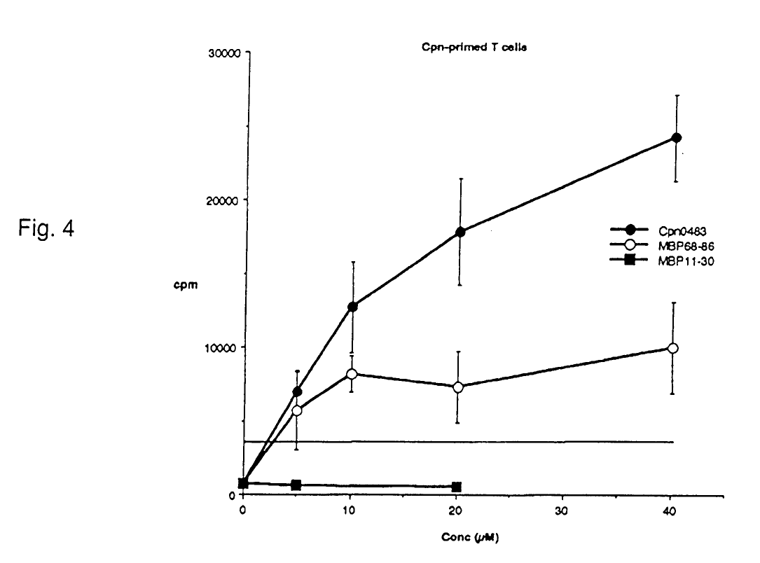

A preferred group of Cpn0483 peptide variants are those in which at least one amino acid residue and preferably, only one, has been substituted by different residue. For a detailed description of protein chemistry and structure, see Schulz, G E et al., Principles of Protein Structure, Springer-Verlag, New York, 1978, and Creighton, T. E., Proteins: Structure and Molecular Properties, W. H. Freeman & Co., San Francisco, 1983, which are hereby incorporated by reference. The types of substitutions that may be made in the protein molecule may be based on analysis of the frequencies of amino acid changes between a homologous protein of different species, such as those presented in Table 1-2 of Schulz et al. (supra) and FIGS. 3-9 of Creighton (supra). Based on such an analysis, conservative substitutions are defined herein as exchanges within one of the following five groups:

| | | |
|---|---|---|
| 1 | Small aliphatic, nonpolar or | Ala, Ser, Thr (Pro, Gly); |
| | slightly polar residues | |
| 2 | Polar, negatively charged residues | Asp, Asn, Glu, Gln; |
| | and their amides | |
| 3 | Polar, positively charged residues | His, Arg, Lys; |
| 4 | Large aliphatic, nonpolar residues | Met, Leu, Ile, Val (Cys) |
| 5 | Large aromatic residues | Phe, Tyr, Trp. |

The three amino acid residues in parentheses above have special roles in protein architecture. Gly, the only residue lacking a side chain, imparts flexibility to the chain. Pro, because of its unusual geometry, tightly constrains the chain. Cys can participate in disulfide bond formation which is important in protein folding.

More substantial changes in biochemical, functional (or immunological) properties are made by selecting substitutions that are less conservative, such as between, rather than within, the above five groups. Such changes will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain. Examples of such substitutions are (i) substitution of Gly and/or Pro by another amino acid or deletion or insertion of Gly or Pro; (ii) substitution of a hydrophilic residue, e.g., Ser or Thr, for (or by) a hydrophobic residue, e.g., Leu, Ile, Phe, Val or Ala; (iii) substitution of a Cys residue for (or by) any other residue; (iv) substitution of a residue having an electropositive side chain, e.g., Lys, Arg or His, for (or by) a residue having an electronegative charge, e.g., Glu or Asp; or (v) substitution of a residue having a bulky side chain, e.g., Phe, for (or by) a residue not having such a side chain, e.g., Gly.

Most acceptable deletions, insertions and substitutions according to the present invention are those that do not produce radical changes in the characteristics of the peptide in terms of its biological or binding activity as described herein. However, when it is difficult to predict the exact effect of the substitution, deletion or insertion in advance of doing so, one skilled in the art will appreciate that the effect can be evaluated by routine screening assays such as those described here, without requiring undue experimentation.

Among the addition variants contemplated are fusion proteins comprising the Cpn0483 peptide (or functional derivative or mimetic) that is fused to another peptide or polypeptide that confers useful properties on the peptide.

A “chemical derivative” of the peptide of the present invention contains additional chemical moieties not normally a part of the peptide. Covalent modifications of the peptide are included within the scope of this invention. Such modifications may be introduced into the molecule by reacting targeted amino acid residues of the peptide with an organic derivatizing agent that is capable of reacting with selected side chains or terminal residues.

Lysinyl and amino terminal residues are reacted with succinic or other carboxylic acid anhydrides. Derivatization with these agents has the effect of reversing the charge of the lysinyl residues. Other suitable reagents for derivatizing I-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Carboxyl side groups (aspartyl or glutamyl) are selectively modified by reaction with carbodiimides (R′—N—C—N—R′) such as 1-cyclohexyl-3-(2-morpholinyl-(4-ethyl)carbodiimide or 1-ethyl-3(4-azonia 4,4-dimethylpentyl)carbodiimide. Aspartyl and glutamyl residues can be converted to asparaginyl and glutaminyl residues by reaction with ammonium ions.

The specific modification of tyrosyl residues can be by introducing spectral labels by reaction with aromatic diazonium compounds or tetranitromethane. Most commonly, N-acetylimidizol and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, such as by the chloramine T method.

Derivatization with bifunctional agents is useful for crosslinking the protein or peptide molecule, such as to a water-insoluble support matrix or surface. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues may be deamidated to the corresponding glutamyl and aspartyl residues. Alternatively, these residues are deamidated under mildly acidic conditions.

Other modifications include hydroxylation of proline and lysine, phosphorylation of hydroxyl groups of seryl or threonyl residues, methylation of the I-amino groups of lysine, arginine, and histidine side chains (T. E. Creighton, Proteins: Structure and Molecule Properties, W. H. Freeman & Co., San Francisco, pp. 79-86 (1983)), acetylation of the N-terminal amine, and, in some instances, amidation of the C-terminal carboxyl groups.

Also included in the scope of the invention are salts of the peptides of the invention. As used herein, the term “salts” refers to both salts of carboxyl groups and to acid addition salts of amino groups of the protein or peptide molecule. Salts of a carboxyl group may be formed by means known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuric acid, and salts with organic acids such as, for example, acetic acid or oxalic acid.

It is also understood that enzymatic degradation of the proteins or peptides of the present invention in vivo may cause the peptides to be relatively short-lived. One method of preventing such degradation would be by making synthetic peptides containing a D-amino acid.

Another modification involves extending the peptide by moieties intended to affect solubility, e.g., by the addition of a hydrophilic residue, such as serine, or a charged residue, such as glutamic acid. Furthermore, the peptide could be extended for the purpose of stabilization and preservation of a desired conformation, such as by adding cysteine residues for the formation of disulfide bridges.

Another reason to modify the peptides would be to permit their detection after administration. This can be done by radioiodination (e.g., at the tyrosine residue) with a radioactive iodine isotope, directly, or by first adding one or more tyrosines before radioiodination.

Peptidomimetics

A preferred type of chemical derivative of the peptides described herein is a peptidomimetic compound which mimics the biological effects of the Cpn0483 peptide. A peptidomimetic agent may be an unnatural peptide or a non-peptide agent which recreates the stereospatial properties of the binding elements of the Cpn0483 peptide such that it has the binding activity or biological activity of the Cpn0483 peptide. Similar to the Cpn0483 peptide, a peptidomimetic will have a binding face (which interacts with, for example the relevant TCR) and a non-binding face. Again, similar to the Cpn0483 peptide, the non-binding face of a peptidomimetic will contain functional groups which can be modified by various therapeutic or diagnostic moieties without modifying the binding face of the peptidomimetic. A preferred embodiment of a peptidomimetic would contain an aniline on the non-binding face of the molecule. The NH₂-group of an aniline has a pKa˜4.5 and could therefore be modified by any NH₂-selective reagent without modifying any NH₂ functional groups on the binding face of the peptidomimetic. Other peptidomimetics may not have any NH₂ functional groups on their binding face and therefore, any NH₂, without regard for pKₐ could be displayed on the non-binding face as a site for conjugation. In addition other modifiable functional groups, such as —SH and —COOH could be incorporated into the non-binding face of a peptidomimetic as a site of conjugation. A therapeutic or diagnostic moiety could also be directly incorporated during the synthesis of a peptidomimetic and preferentially be displayed on the non-binding face of the molecule.

This invention also includes compounds which retain partial peptide characteristics. For example, any proteolytically unstable bond within the Cpn0483 peptide could be selectively replaced by a non-peptidic element such as an isostere (N-methylation; D-amino acid) or a reduced peptide bond while the rest of the molecule retains its peptide nature.

Peptidomimetic compounds, either agonists, substrates or inhibitors, have been described for a number of bioactive peptides such as opioid peptides, VIP, thrombin, HIV protease, etc. Methods for designing and preparing peptidomimetic compounds are known in the art (Hruby, V. J., Biopolymers 33:1073-1082 (1993); Wiley, R. A. et al., Med. Res. Rev. 13:327-384 (1993); Moore et al., Adv. in Pharmacol 33:91-141 (1995); Giannis et al., Adv. in Drug Res. 29:1-78 (1997), which references are incorporated by reference in their entirety). These methods are used to make peptidomimetics that possess at least the binding capacity and specificity of the cyclic peptides and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the cystallographically-derived three-dimensional structure of a peptide of the invention either free or bound in complex with the binding groove of MHC class II protein or with a the TCR V region protein or peptide. Alternatively, the structure of a peptide of the invention bound to its ligand an be gained by the techniques of nuclear magnetic resonance spectroscopy. The better knowledge of the stereochemistry of the interaction of a peptide with its receptor or binding partner will permit the rational design of such peptidomimetic agents.

One requirement for a protein, peptide, peptidomimetic antibody to serve as an inhibitor of T lymphocyte activation or function in accordance with the present invention is that it be a ligand for the TCR and/or an MHC molecule. Recognition of this peptide is preferably such that the peptide is bound by a Cpn0483 peptide-specific T cell, or with the appropriate MHC molecule on or in an antigen presenting cell, with sufficient affinity to compete successfully for binding with a native or other stimulatory Cpn0483 peptide or MBP peptide. Alternatively, the inhibitory moiety should bind with sufficient affinity to an anti-Cpn0483 peptide antibody to inhibit the antibody from binding to cells or tissue.

Peptide-MHC Complexes

The present invention includes multi-molecular “complexes” which can be used to modulate T cell function. For instance, the complexes can be used to inhibit a deleterious T cell-mediated immune response, such as a pathogenic autoimmune response leading to a demyelinating diseases, preferably MS. In addition, these complexes can also be used as vaccines that promote T cell responses.

The complex of the invention includes at least two components: (1) the Cpn peptide or functional derivative as described herein which represents an autoantigen or other antigenic sequence with an effect on the immune system in the context of EAE and/or MS, and (2) an effective portion of an MHC-encoded glycoprotein involved in antigen presentation. An effective portion of an MHC glycoprotein is one which comprises a binding site or groove for the peptide (“antigen binding site”) and a sequence(s) involved in T cell recognition of the MHC-peptide complex by the appropriate TCR. The MHC component can be either a Class I or a Class II molecule. The nature of the bonding between the peptide and the antigen binding sites of the MHC protein can be by covalent or by noncovalent bonding.

In other embodiments the complex may also contain an effector component such as a toxin or a detectable label. The effector portion may be conjugated to either the MHC glycoprotein or to the Cpn peptide.

The MHC Component

The glycoproteins encoded by the MHC have been extensively studied in both the human and murine systems. In general, they have been classified as Class I glycoproteins, found on the surfaces of all cells and primarily recognized by cytotoxic T cells; and Class II glycoproteins which are expressed on a limited range of cells, including accessory cells or antigen presenting cells (APC) such as dendritic cells (DC) and macrophages. A number of MHC proteins of both classes have been isolated and characterized. For a general review of MHC glycoprotein structure and function, see A. K. Abbas et al., Cellular and Molecular Immunology (Fourth Ed.), W. B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C. V. Mosby Co., St. Louis, Mo. (1999), which are incorporated herein by reference. The term “isolated MHC component” refers to an MHC glycoprotein or an immunologically effective portion of an MHC glycoprotein (i.e., one that comprises an antigen binding site/sites and sequences necessary for TCR recognition) which is in other than its native state, for example, not associated with the cell membrane. The MHC component may be recombinantly produced, solubilized from an appropriate cell source or embedded in a liposome.

Methods for purifying murine class II MHC proteins are well known (e.g., Turkewitz, A. P. et al., Mol. Immunol. (1983) 20:1139-1147) and are generally suitable for Class I MHC molecules. These approaches start from the preparation of a soluble membrane extracts from cells using nonionic detergents. The MHC molecules are then purified by affinity chromatography using specific antibodies raised as affinity reagents. Isolated murine class II proteins encoded by the I-A and I-E genes are heterodimers of two noncovalently bonded peptide chains: an α chain of 32-38 kDa and a β chain of 26-29 kDa. A third, invariant chain (31 kDa) is noncovalently associated but is not polymorphic and is generally not found on the cell surface.

The chemistry and crystal structure of human class I and class II proteins are well known in the art (e.g., Bjorkman, P J. et al., Nature (1987) 329:506-512, 512-518). Class II glycoproteins have a domain structure, including an antigen binding site, similar to that of Class I. It is formed from the N-terminal domain portions of two class II chains which extend from the membrane bilayer. The N-terminal portion of one chain has two domains of homology with the α1 and α2 regions of the MHC Class I antigen sequence. The MHC glycoproteins for making the present complexes can be obtained from human cells, e.g., B cells or cell lines and are screened peptide binding using conventional assays. Alternatively, as the amino acid sequences of these MHC proteins are known, and their DNA has been cloned, recombinant methods may be used for preparing these proteins.

Antigenic Peptide

The antigenic peptides of interest are described in detail herein. The part of the peptide important for association with the MHC class I or class II peptide binding groove is about 8-15 residues in length, and contains both the “agretope” (recognized by the MHC molecule) and the epitope (recognized by TCR).

A set of labeled test peptides can be prepared, and those which bind to MHC in planar lipid membranes containing MHC proteins are considered to include the agretope.

Complex Formation

The elements of the complex can be associated by standard means. The peptides can be associated noncovalently with the pocket portion of the MHC protein by, for example, mixing the two components. They can also be covalently bound by, for example, photo affinity labelling, (Hall et al., Biochemistry 24:5702-5711 (1985)). For example the Cpn0483 peptide, SEQ ID NO: 3, may be bonded to the N-terminal antigen binding site of a polypeptide derived from an MHC antigen associated with MS. An oligonucleotide which encodes the peptide is synthesized using the known codons for the amino acid, preferably those codons which have preferred utilization in the organism which is to be used for expression are utilized in designing the oligonucleotide. This sequence may then be incorporated into a sequence encoding the MHC class II sequence utilizing techniques known in the art. When the molecule is expressed and folded, the peptide will be available as an epitope for the relevant T cells.

In one protocol, the Cpn0483 peptide is bonded to the N-terminus of an α or β chain of an appropriate MHC class II molecule (i.e., the DR allele corresponding to the MHC allele of the subject). In one approach the peptide is a replacement for the leader peptide. Methods of replacing sequences within polynucleotides are known in the art. In addition to peptidic linkages, the peptide may be linked to the MHC glycoprotein via carbohydrate groups

Assessment of the Complexes

The complexes of the invention can be assayed using an in vitro system or using an in vivo model. For in vitro analysis the complex is incubated with peripheral blood T cells from subjects immunized with, or showing immunity to MBP, or to the Cpn peptide itself. A successful complex will induce anergy in such T cells or otherwise suppress or prevent their proliferation even in the presence of a stimulatory form of the autoantigen. In in vivo, T cells that proliferate in response to the autoantigen (e.g., MBP) in the presence of APC may be cloned, the clones tested in vitro as above or, if practical, injected into a histocompatible non-immune animal to induce EAE. The complex is tested for its ability to protect the animal from the encephalitogenic treatment or to treat the diseases once it has been induced.

In treatment approach, the subject is treated with the MHC-peptide complex to down-regulate the pathogenic immune response. Further down-regulation is achieved by treating with a three component complex of MHC component, peptide, and an effector component. “Panels” of complexes may be used in the case when more than one epitope is known be involved in the immune response and, thereby, in disease pathogenesis. In this context, the same MHC proteins may be combined with different peptides, or different combinations used. Use of detectably labeled complexes permit localization of the complex in vivo after its administration and thereby focus on those sites in which immune reactivity and its suppression or reversal are occurring. This may also have diagnostic or prognostic implications.

Selection of the MHC for use in Complexes

MHC molecules or components for use in the present complexes are selected based on the subject's genotype and/or the particular association between MHC genotype and disease susceptibility.

Formulation and Administration of Complexes

If the transmembrane region of the MHC subunit is included, the complexes are conveniently administered after incorporation into lipid monolayers or bilayers. Typically liposomes are used for this purpose but any form of lipid membrane, such as planar lipid membranes or the cell membranes (e.g., red blood cell) may be used. The complexes are also conveniently incorporated into micelles. MHC-peptide complexes comprising dimeric MHC molecules are expected to exist primarily as aggregates.

Liposomes can be prepared according to standard methods. If the transmembrane region of the MHC component is deleted, the complex can be incorporated into liposomes in the same way that this is done with peptide or polypeptide pharmaceuticals. General aspects of formulation and administration of pharmaceutical compositions herein is discussed in a section below.

Degeneracy of T Cell Receptor Recognition

A role for degenerate T cell recognition has been postulated for such diverse immunological phenomena as thymic selection, peripheral T cell survival, protection from infectious diseases, and induction of autoimmunity (Hemmer, B et al., 1998, Immunol. Today 19:163,; Gran, B. et al., 1999 “Molecular mimicry and multiple sclerosis: degenerate T-cell recognition and the induction of autoimmunity.” Ann. Neurol. 45:559. Peptide combinatorial libraries in the positional scanning format were known to be useful to define the spectrum of agonist ligands for clonotypic TCR.

A recent study published by Zhao Y et al., J. Immunol, 2001 167:2130-2141, shows that interaction of TCRs with MHC peptide ligands can be highly flexible, so that many different peptides are recognized by the same TCR in the context of a single restriction element. This is also referred to as T cell degeneracy. The authors provided a quantitative analysis of such interactions that permits one to identify T cell epitopes and molecular mimics. The response of T cell clones to positional scanning synthetic combinatorial libraries was analyzed mathematically based on a model that assesses the independent contribution of individual amino acids to peptide antigen recognition. The analysis carried out by these authors compared the information derived from libraries composed of trillions of decapeptides with all the millions of decapeptides contained in a protein database and ranked and predicted the most stimulatory peptides for a given T cell clone. The authors demonstrated how the search results from their prediction strategy can be related to tissue-specific expression profiles determined by cDNA microarrays to identify candidate peptides that are derived from proteins that are overexpressed in a diseased tissue, such as the brain in MS and are thus available for the expansion of autoreactive T cells.

The predictions based on this methodology are so accurate that they actually lend strong support to an additive, combinatorial model of peptide antigenicity. Available TCR crystal structures indeed suggest that peptides may modulate the preexisting affinity between MHC and TCR that is based on a large contact surface between these two components of the trimolecular complex (Garboczi, D N et al., 1996, Nature 384:134; Garcia, K C et al., 1996, Science 274:209). This model was found to extends and develop the concept of primary and secondary TCR contacts (Kersh, G J P et al., 1996, J. Exp. Med. 184:1259; Degano, M et al., 2000, Immunity 12:251). Although complex substitutions of amino acids along the entire sequence of the peptide can lead to molecular mimicry in the absence of any sequence homology, the relative weight of different amino acids in each position of the peptides sequence was shown to be apparent from the results.

The above method may be used in the context of the present invention to predict structural changes in autoantigenic peptides and/or in the Cpn peptides described herein that can permit selection of the best peptides to use to treat a subject whose T cell reactivity is oriented to a particular autoantigenic epitope.

Antibodies Specific for the Cpn0483 Peptide

In the following description, reference will be made to various methodologies known to those of skill in the art of immunology, cell biology, and molecular biology. Publications and other materials setting forth such known methodologies to which reference is made are incorporated herein by reference in their entireties as though set forth in full. Standard reference works setting forth the general principles of immunology include A. K. Abbas et al., Cellular and Molecular Immunology (Fourth Ed.), W. B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C. V. Mosby Co., St. Louis, Mo. (1999); Klein, J., Immunology, Blackwell Scientific Publications, Inc., Cambridge, Mass., (1990).

Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Immunoassay methods are also described in Coligan, J. E. et al., eds., Current Protocols in Immunology, Wiley-Interscience, New York 1991(or current edition); Butt, W. R. (ed.) Practical Immunoassay: The State of the Art, Dekker, New York, 1984; Bizollon, Ch. A., ed., Monoclonal Antibodies and New Trends in Immunoassays, Elsevier, New York, 1984; Butler, J. E., ELISA (Chapter 29), In: van Oss, C. J. et al., (eds), IMMUNOCHEMISTRY, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T. S. et al., Laboratory Techniques and Biochemistry in Molecular Biology, North Holland Publishing Company, NY, (1978) (Chapter by Chard, T., “An Introduction to Radioimmune Assay and Related Techniques”).

Anti-idiotypic antibodies are described, for example, in Idiotypy in Biology and Medicine, Academic Press, New York, 1984; Immunological Reviews Volume 79, 1984; Immunological Reviews Volume 90, 1986; Curr. Top. Microbiol., Immunol. Volume 119, 1985; Bona, C. et al., CRC Crit. Rev. Immunol., pp. 33-81 (1981); Jerne, N K, Ann. Immunol. 125C:373-389 (1974); Jerne, N K, In: Idiotypes—Antigens on the Inside, Westen-Schnurr, I., ed., Editiones Roche, Basel, 1982, Urbain, J et al., Ann. Immunol. 133D:179-(1982); Rajewsky, K. et al., Ann. Rev. Immunol. 1:569-607 (1983).

The present invention provides antibodies, both polyclonal and monoclonal, reactive with novel epitopes of the Cpn0483 protein SEQ ID NO: 2, and preferably an epitope or epitope present in the peptide SEQ ID NO: 3 or to a functional derivative thereof, such as a substitution variant. As used herein, an antibody to the peptide is intended to include an antibody to such a derivative. The antibodies may be produced in any mammal or may be modified forms, such as humanized or chimeric antibodies. Antiidiotypic antibodies specific for the idiotype of an anti- the Cpn0483 peptide antibody are also included. The term “antibody” is also meant to include both intact molecules as well as fragments thereof that include the antigen-binding site and are capable of binding to the Cpn0483 peptide. These include, Fab and F(ab′)₂ fragments which lack the Fc fragment of an intact antibody, clear more rapidly from the circulation, and may have less non-specific tissue binding than an intact antibody (Wahl et al., J. Nucl. Med. 24:316-325 (1983)). Also included are Fv fragments (Hochman, J. et al. (1973) Biochemistry 12:1130-1135; Sharon, J. et al.(1976) Biochemistry 15:1591-1594).). These various fragments are be produced using conventional techniques such as protease cleavage or chemical cleavage (see, e.g., Rousseaux et al., Meth. Enzymol., 121:663-69 (1986)).

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography (see Zola et al., supra).

The immunogen may comprise the complete the Cpn0483 protein, or a fragments or derivatives thereof that includes SEQ ID NO: 3 or a variant thereof against which an antibody is desired.

The mAbs may be produced using conventional hybridoma technology, such as the procedures introduced by Kohler and Milstein (Nature, 256:495-97 (1975)),-and modifications thereof (see above references). An animal, preferably a mouse is primed by immunization with an immunogen as above to elicit the desired antibody response in the primed animal.

B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using the Cpn0483 peptide-Ig fusion protein Positive clones are subcloned, e.g., by limiting dilution, and the mAbs are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single mAb can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost C R et al,. J Biol Chem. 1994 269:26267-26273; U.S. Pat. Nos. 4,704,692, 4,853,871, 4,94,6778, 5,260,203, 5,455,030.

Typical, and preferred, immunometric assays include “forward” assays in which the antibody bound to the solid phase is first contacted with the sample being tested to extract the antigen from the sample by formation of a binary solid phase antibody-antigen complex. After a suitable incubation period, the solid support is washed to remove the residue of the fluid sample, including unreacted antigen, if any, and then contacted with the solution containing an unknown quantity of labeled antibody (which functions as a “reporter molecule”). After a second incubation period to permit the labeled antibody to complex with the antigen bound to the solid support through the unlabeled antibody, the solid support is washed a second time to remove the unreacted labeled antibody. This type of forward sandwich assay may be a simple “yes/no” assay to determine whether antigen is present or may be made quantitative by comparing the measure of labeled antibody with that obtained for a standard sample containing known quantities of antigen.

In another type of “sandwich” assay the so-called “simultaneous” and “reverse” assays are used. A simultaneous assay involves a single incubation step as the antibody bound to the solid support and labeled antibody are both added to the sample being tested at the same time. After the incubation is completed, the solid support is washed to remove the residue of fluid sample and uncomplexed labeled antibody. The presence of labeled antibody associated with the solid support is then determined as it would be in a conventional “forward” sandwich assay.

In the “reverse” assay, stepwise addition first of a solution of labeled antibody to the fluid sample followed by the addition of unlabeled antibody bound to a solid support after a suitable incubation period is utilized. After a second incubation, the solid phase is washed in conventional fashion to free it of the residue of the sample being tested and the solution of unreacted labeled antibody. The determination of labeled antibody associated with a solid support is then determined as in the “simultaneous” and “forward” assays.

The foregoing antibodies are useful in method for inhibiting T cell stimulation and treating diseases associated with undesired activation of T cells reactive with the Cpn0483 peptide such as T cells involve in rodent EAE or human MS. This method involves administering a subject in need of such treatment an effective amount of an antibody, preferably a mAb, more preferably a human or humanized mAb specific for epitope of the Cpn0483 peptide. The administration of antibody must be effective in blocking stimulation of T cells, in eliminating antigen-reactive T cells, or otherwise in preventing or inhibiting the action of such T cells. Relevant dose ranges are described below.

Generation of, and Epitope Recognition by Cpn0483 Peptide Specific T Cell Lines (TCL) and T-Cell Clones (TCC)

PBL are incubated in complete medium (see Examples) at densities likely to contain a single antigen specific cell. Cells are stimulated for 1 week with the Cpn0483 peptide antigen at concentrations readily ascertainable by those of skill in the art. Interleukin 2 (IL-2) can be added to promote expansion of antigen-specific cells. On about day 8, and subsequently at about weekly intervals, fresh medium is added with 10⁴ irradiated antigen-presenting cells (APC), 50 U/ml rIL2, and antigen. T cell lines are stimulated repeatedly until their response to the Cpn0483 peptide or MBP (or an MBP peptide) equals or exceeds their response to the polyclonal T cell activator, Con A. Established TCLs are cloned by limiting dilution at 0.5 cell/well in 96-well plates with the use of 5 μg/ml ConA and 10⁵ irradiated autologous peripheral blood mononuclear cells (PBMC) as feeder cells. About 50 U/ml IL-2 is added on day 3 and cells are fed bi-weekly with antigen and IL-2 containing medium. On day 14, cultures showing positive growth are expanded by restimulation with antigen, IL-2, and autologous feeder cells. T cell clones are retested for antigen-specificity in proliferation assays in the presence of APC (autologous Epstein-Barr virus-transformed B cells or irradiated PBMC), the Cpn0483 peptide-specific T cells preferably maintained by stimulation with 50 U/ml IL-2, 5 μg/ml Cpn0483 peptide, and 10⁵ irradiated PBMC.

Epitope specificity of the Cpn0483 peptide-responsive T cell lines is determined using a panel of overlapping synthetic peptides as described above. Because pathogenesis of MS is likely to involve several autoantigens and a heterogeneous population of T cells, identification of immunodominant T cell epitopes is important for the development of antigen-analog peptide vaccines for MS. T cell responsiveness is assessed generally as described in the examples for PBL, though lower numbers of cells are used. Thus, about 2×10⁴ T cells are stimulated with 10 and 100 μg/ml synthetic Cpn0483 peptide in the presence of APC for 72 h and ³HTdR incorporation measured. The number of T cell lines responding to each epitope should reflect the relative frequency of these clones in the peripheral blood and CSF. Therefore, peptides representing immunodominant epitopes are used to stimulate freshly isolated T cells from peripheral blood and CSF and compared to the proliferative response to full length recombinant the Cpn0483 peptide.

T Cell Receptor (TCR) Repertoire of Cpn0483 Peptide Reactive T Cells

The Cpn0483 peptide at concentrations as low as 1 μg/ml is expected to significantly stimulate the proliferation of PBL of MS patients. Knowledge of the clonality of the T cell response to the Cpn0483 peptide or one of its epitopes is important for designing T cell-directed therapeutic interventions. According to the present invention, the TCR usage is determined by examination of Vα and Vβ-expressing T cell subsets upon stimulation of T cell proliferation by recombinant the Cpn0483 peptide or its immunodominant peptides in comparison to an unrelated antigen such as tetanus toxoid, or polyclonal stimulation by an anti-CD3 mAb. Staphylococcal enterotoxin B, the superantigen of Staphylococcus aureus (Sigma) is used as a positive control for induction of Vβ-specific T cells (Kappler, J. et al., 1989, Science 244:811-813).

Before and after stimulation with antigens (or anti-CD3 mAb) for about 72 h, total RNA is be prepared from each T cell culture and analyzed for Vα and Vβ expression by reverse transcriptase mediated PCR (RT-PCR)(Genevee, C. et al., 1992, Eur. J. Immunol. 22:1261-1269). If expanded clones dominate the response to immunodominant epitopes in a given patient, most Cpn0483 peptide-specific clones independently generated from that patient will have identical TCR rearrangements. The characterization of such autoreactive clones will not only help to understand the pathogenesis of MS but will also assist in the design of TCR V gene-specific immunotherapy (see, for example, Vandenbark, A. A. et al., 1993, Inter. Rev. Immunol. 9:251-276; Bourdette, D. N. et al., 1994, J Immunol. 152:2510-2519; Chou, Y. K. et al., J. Immunol. 152:2520-2529).

Therapeutic Use of Peptides, Complexes, or Antibodies of the Invention

As mentioned above, the compositions of the present invention are useful in the therapy of MS or other neurodegenerative diseases associated with Cpn0483 peptide-specific autoimmune reactivity. The therapeutic embodiments of the present invention based on the association between the disease, such as MS, and the presence of anti-Cpn0483 peptide antibodies and/or the Cpn0483 peptide-specific T cell immunity. Targeted removal or diminution of the concentration of such antibodies or T cells are expected to alleviate symptoms of or progression of the disease, possibly inducing remission.

The Cpn0483 peptide proteins, peptide or functional derivative preparations are therapeutically useful in part because they may interfere with the binding of T cells via their TCRs to the MHC/antigen complex needed for initiation or propagation of the immune recognition or inflammatory process underlying MS.

The therapeutic peptide peptides according to the present invention are administered to patients having, or known to be susceptible to, an immune-related disease neurodegenerative disease, particularly, MS, in amounts sufficient to protect the patient from the disease by preventing the patient's immune system from activation leading to induction, maintenance or exacerbation of the disease state.

The route of administration are preferably intravenous, subcutaneous, intramuscular or intrathecal routes. Alternatively, or contemporaneously, the agents may be given by any or thee following routes: inhalation, intraperitoneal, intranasal, intraarticular, intradermal, transdermal or other known routes.

The therapeutic use of the present invention in the treatment of disease or disorders will be best accomplished by those of skill, employing accepted principles of treatment. Such principles are known in the art, and are set forth, for example, in Braunwald, E. et al., eds., Harrison's Principles of Internal Medicine, 11th Ed., McGraw-Hill, New York, N.Y. (1987 or current edition).

The peptides of the present invention or their functional derivatives, are well suited for the preparation of pharmaceutical compositions. The pharmaceutical compositions of the invention may be administered to any animal which may experience the beneficial effects of the compositions of the invention. Foremost among such animals are humans, although the invention is not intended to be so limited.

The pharmaceutical compositions of the present invention may be administered by any means that achieve their intended purpose, for example, by the routes described above. Alternatively, or concurrently, administration may be by the oral route. The peptides and pharmaceutical compositions can be administered parenterally by bolus injection or by gradual perfusion over time.

A therapeutically effective amount is a dosage that, when given for an effective period of time, achieves the desired immunological or clinical effect.

A therapeutically active amount of a polypeptide or peptide composition having the biological activity of the Cpn0483 peptide activity (or of an anti-Cpn0483 peptide antibody) may vary according to factors such as the disease state, age, sex, and weight of the individual, and the ability of the peptide to elicit a desired response in the individual. Dosage regimes may be adjusted to provide the optimum therapeutic response. For example, several divided doses may be administered daily or the dose may be proportionally reduced as indicated by the exigencies of the therapeutic situation. A therapeutically effective amounts of the protein, in cell associated form may be stated in terms of the protein or cell equivalents.

The dose ranges for the administration of the compositions of the present invention are those large enough to produce the desired effect, whereby, for example, an immune response to a stimulatory peptide, such as an MBP peptide, as measured by T cell proliferation in vitro or a delayed hypersensitivity response in vivo, is substantially prevented or inhibited, and further, where the immune-related disease is significantly treated. The doses should not be so large as to cause adverse side effects, such as unwanted cross reactions, generalized immunosuppression, anaphylactic reactions and the like.

Effective doses of a the therapeutic peptide of this invention for use in treating an immune-related disease, particularly MS, are in the range of about 1 ng to 100 mg/kg body weight. A preferred dose range is between about 10 ng and 10 mg/kg. A more preferred dose range is between about 100 ng and 1 mg/kg.

Thus an effective amount of the therapeutic peptide of this invention for use in treating an immune-related disease, particularly MS, is between about 1 ng and about 1 gram per kilogram of body weight of the recipient. A preferred dose range is between about 10 ng and 10 mg/kg. A more preferred dose range is between about 100 ng and 1 mg/kg. Dosage forms suitable for internal administration preferably contain (for the latter dose range) from about 0.1 mg to 500 mg of active ingredient per unit. The active ingredient may vary from 0.5 to 95% by weight based on the total weight of the composition. Alternatively, an effective dose of cells expressing the Cpn0483 protein or peptide, such cells which have been transfected or transduced to express this peptide, is between about 10⁴ and 10⁹ cells, more preferably between about 10⁶ and 10⁸ cells per subject, preferably in split doses. Those skilled in the art of immunotherapy will be able to adjust these doses without undue experimentation.

In addition to peptides of the invention which themselves are pharmacologically active, pharmaceutical compositions may contain suitable pharmaceutically acceptable carriers comprising excipients and auxiliaries which facilitate processing of the active compounds into preparations which can be used pharmaceutically.

The active compound (e.g., the Cpn0483 peptide polypeptide or cell transduced with the Cpn0483 peptide DNA) may be administered in a convenient manner, e.g., injection by a convenient and effective route. Preferred routes include subcutaneous, intradermal, intravenous and intramuscular routes. Other possible routes include oral administration (ingestion), intrathecal, inhalation (preferably intranasal), transdermal application, or rectal administration. For the treatment of tumors which have not been completely resected, direct intratumoral injection is also intended.

Depending on the route of administration, the active compound may be coated in a material to protect the compound from the action of enzymes, acids and other natural conditions which may inactivate the compound. Thus, to a administer a polypeptide or Cpn0483 peptide by an enteric route, it may be necessary to coat the composition with, or co-administer the composition with, a material to prevent its inactivation. For example, a peptide may be administered to an individual in an appropriate carrier, diluent or adjuvant, co-administered with enzyme inhibitors (e.g., pancreatic trypsin inhibitor, diisopropylfluorophosphate (DEP) and trasylol) or in an appropriate carrier such as liposomes (including water-in-oil-in-water emulsions as well as conventional liposomes (Strejan et al., (1984) J. Neuroimmunol 7:27). Other coatings known in the art are described, for example, in: K. Lehman, Acrylic Coatings in Controlled Release Tablet Manufacturer, Manufacturing Chemist and Aerosol News, June 1973), and K. Lehman, Programmed Drug Release From Oral Program Forms; Pharma. Int., vol. ISS 3 1971, p. 34-41. The latter documents describe enteric coatings such as Eudragit S and Eudragit L. See also, “Handbook of Pharmaceutical Excipients, most current edition.

As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

Preferred physiologically or pharmaceutically acceptable diluents, carrier or excipients may comprise buffers such as neutral buffered saline, phosphate buffered saline and the like, carbohydrates such as glucose, mannose, sucrose or dextrans, mannitol, proteins, polypeptides or amino acids such as glycine, antioxidants, chelating agents such as EDTA or glutathione. Pharmaceutical compositions suitable for injection include sterile aqueous solutions (where water soluble) or dispersions and sterile powders for the extemporaneous preparation of sterile injectable solutions or dispersion. In all cases, the composition should be sterile and should be fluid. It should be stable under the conditions of manufacture and storage and must include preservatives that prevent contamination with microorganisms such as bacteria and fungi.

In certain embodiments, and when stimulation of an immune response or of certain T lymphocyte populations is desired, an adjuvant (e.g., aluminum hydroxide or any other acceptable adjuvant) may be used. Additional active ingredients, such as, for example, an appropriately stimulatory cytokine. For inducing regulatory Th2 cells in vitro or in vivo, which cells are either protective or therapeutic in MS, co-administration of IL-4 is preferred to drive the activation of these particular T cells.

Dispersions can also be prepared in glycerol, liquid polyethylene glycols, and mixtures thereof and in oils. Under ordinary conditions of storage and use, these preparations may contain a preservative to prevent the growth of microorganisms.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal, and the like.

Prolonged absorption of the injectable compositions can be brought about by including in the composition an agent which delays absorption, for example, aluminum monostearate and gelatin.

Parenteral compositions are preferably formulated in dosage unit form for ease of administration and uniformity of dosage. Dosage unit form refers to physically discrete units suited as unitary dosages for a mammalian subject; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For lung instillation, aerosolized solutions are used. In a sprayable aerosol preparations, the active protein may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

For topical application, the proteins of the present invention may be incorporated into topically applied vehicles such as salves or ointments, which have both a soothing effect on the skin as well as a means for administering the active ingredient directly to the affected area.

The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Examples of preferred vehicles for non-sprayable topical preparations include ointment bases, e.g., polyethylene glycol-1000 (PEG-1000); conventional creams such as HEB cream; gels; as well as petroleum jelly and the like.

Other pharmaceutically acceptable carriers for the Cpn0483 peptide or polypeptide according to the present invention are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

To enhance delivery or bioactivity, the peptides can be incorporated into liposomes using methods and compounds known in the art.

Preparations which can be administered orally in the form of tablets and capsules, preparations which can be administered rectally, such as suppositories, and preparations in the form of solutions for injection or oral introduction, contain from about 0.001 to about 99 percent, preferably from about 0.01 to about 95 percent of active compound(s), together with the excipient.

Suitable formulations for parenteral administration include aqueous solutions of the peptides in water-soluble form, for example, water-soluble salts. In addition, suspensions of the peptides as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers.

Peptide analogs of the present invention may be administered either alone, or as a pharmaceutical composition. Briefly, pharmaceutical compositions of the present invention may comprise one or more of the peptide analogs described herein, in combination with one or more pharmaceutically or physiologically acceptable carriers, diluents or excipients. Such compositions may comprise buffers such as neutral buffered saline, phosphate buffered saline and the like, carbohydrates such as glucose, mannose, sucrose or dextrans, mannitol, proteins, polypeptides or amino acids such as glycine, antioxidants, chelating agents such as EDTA or glutathione, adjuvants (e.g., aluminum hydroxide) and preservatives. In addition, pharmaceutical compositions of the present invention may also contain one or more additional active ingredients, such as, for example, cytokines like .beta.-interferon.

The peptides are formulated using conventional pharmaceutically acceptable parenteral vehicles for administration by injection. These vehicles are nontoxic and therapeutic, and a number of formulations are set forth in Remington's Pharmaceutical Sciences, Mack Publishing Company, Easton Pa. (Gennaro 18th ed. 1990) (or current edition). Nonlimiting examples of excipients are water, saline, Ringer's solution, dextrose solution and Hank's balanced salt solution. Formulations according to the invention may also contain minor amounts of additives such as substances that maintain isotonicity, physiological pH, and stability.

The peptides of the invention are preferably formulated in purified form substantially free of aggregates and other undesired materials, preferably at concentrations of about 1.0 ng/ml to 100 mg/ml.

### EXAMPLE I

Expression of Cpn0483 in C. pneumoniae.

For RT-PCR analyses, pure RNA was prepared as described (Med. Microbiol. Immunol. 187, 23; 1998) from Hep-2 cells infected with C. pneumoniae strain TW-183; in vitro infection was done by the standard method (e.g., Infect. Immun. 66, 5067; 1998). RNA so prepared was reversed transcribed using the MuLV enzyme (Life Systems, Gaithersburg Md.) and random hexamers as primers. cDNA was purified from the reaction mixtures by extensive treatment with RNAses A, T1, and H, followed by phenol:chloroform extraction and precipitation in ethanol. Amplification of cDNA from infected cultures was done as described for the C. pneumoniae KDO transferase gene and for primary transcripts from the chlamydial rRNA operons (Microb. Pathogen. 29:17, 2000). The primer system used for the mRNA from coding sequence Cpn0483 were:

(SEQ ID NO:22)

| | |
|---|---|
| outer, | 5′-ctgtatgcagtttctac gagctactttc-3′ and |

(SEQ ID NO:23)

5′-cggattaagaagatacgagcgtctc-3′;

(SEQ ID NO:24)

| | |
|---|---|
| inner | 5′-aagactccgctagctg ctcgattagac-3′ and |

(SEQ ID NO:25)

5′-gtcatagcagcgacgtcgttagcct-3′.

Amplification using the inner primer system gives a product of 319 bp. Products were displayed on standard agarose gels and visualized by staining with ethidium bromide. The amplification product was also cloned and the DNA sequence determined to verify its authenticity.

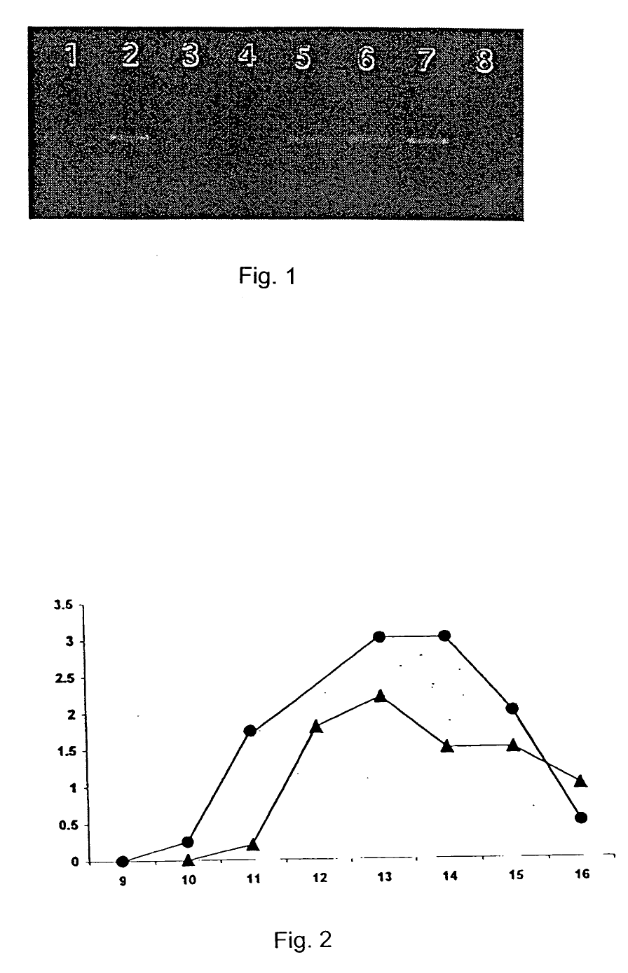

The RT-PCR analyses shown given in FIG. 1 indicates that Cpn0483 is expressed by the bacterium during normal vegetative growth using Hep-2 cells as host in vitro. Transcripts from the Cpn0483 coding sequence are apparent in samples taken 24 hr post-infection, and expression of the gene continues unabated through 72 hr post-infection in this system. Uninfected control cells showed no signal in parallel RT-PCR assays, as expected, and RNA prepared from C. pneumoniae elementary bodies was also negative for this transcript.

### EXAMPLE II

Encephalitogenic Activity in Animals of the Cpn0483 Peptide

The MBP68-86 homolog peptide from Cpn0483 as well as the rat and guinea pig peptides were synthesized using F-moc chemistry in an Applied Biosystems Synergy model 432A Peptide synthesizer (Perkin Elmer, Foster City, Calif.), according to manufacturer's instructions. Peptide structure was confirmed by electrospray mass spectrometry, and purity was determined using HPLC. The peptides used in various of these experiments of this and later Examples are:

| Cpn0483 | RFPNHYGCLLPRNPRTEDQN | SEQ ID NO:3 |
|---|---|---|
| peptide: | | |
| Substituted | | |
| Cpn0483 | | |
| Cpn D>A: | RFPNHYGCLLPRNPRTE [symbol] QN | SEQ ID NO:4 |
| Cpn R>A | RFPNHYGCLLPRNP [symbol] TEDQN | SEQ ID NO:5 |
| Cpn R>N | RFPNHYGCLLPRNP [symbol] TEDQN | SEQ ID NO:6 |
| Cpn R>E | RFPNHYGCLLPRNP [symbol] TEDQN | SEQ ID NO:7 |
| Cpn N>A | RFPNHYGCLLPRNPRTEDQ [symbol] | SEQ ID NO:8 |
| Cpn N>R | RFPNHYGCLLPRNPRTEDQ [symbol] | SEQ ID NO:9 |
| Cpn N>D | RFPNHYGCLLPRNPRTEDQD | SEQ ID NO:10 |
| Cpn C>S | RFPNHYGSLLPRNPRTEDQN | SEQ ID NO:11 |
| Truncated | | |
| Cpn0483 | | |
| N5—truncated | YGCLLPRNPRTEDQN | SEQ ID NO:12 |
| N2—truncated | PNHYGCLLPRNPRTEDQN | SEQ ID NO:13 |
| C5—truncated | RFPNHYGCLLPRNPR | SEQ ID NO:14 |
| N3/C2— | NHYGCLLPRNPRTED | SEQ ID NO:15 |
| truncated | | |
| N4/C2— | HYGCLLPRNPRTED | SEQ ID NO:16 |
| truncated | | |
| Mammalian MBPs | | |
| Rat MBP 68-86: | YGSLPQKSQRTQDENPV | (SEQ ID NO:18) |
| Rat MBP D>A | YGSLPQKSQRSQ [symbol] ENPV | (SEQ ID NO:19) |
| Rat MBP N>A | YGSLPQKSQRSQDE [symbol] PV | (SEQ ID NO:20) |
| G.pig MBP | YGSLPQKSQRSQDENPV | (SEQ ID NO:21) |
| 68-86 | | |

The MBP peptides were numbered according to the bovine MBP sequence (R. E. Martenson, in Experimental Allergic Encephalomyelitis: A Useful Model for Multiple Sclerosis. Progress in Clinical and Biological Research Vol. 146., E. C. Alvord et al., eds. Alan R. Liss, Inc., New York, 1983).

Peptides were administered subcutaneously to LEW rats at 5 μg or 50 μg doses in the standard manner. Eight- to twelve-week-old female Lewis rats (purchased from Charles River, Raleigh, N.C.) were immunized subcutaneously at the hind footpad with the appropriate synthetic peptide, emulsified in complete Freund's adjuvant (CFA, Difco, Detroit, Mich.). They were observed for clinical signs of EAE, graded as 0 (no disease), 1 (loss of tail tonicity), 2 (hind limb weakness), or 3 (hind limb paralysis), as previously described (R. H. Swanborg et al., in Current Protocols in Immunology, vol. 3., J. E. Coligan et al., Eds., Wiley, New York, 1996). Animals were followed for 21 days post-immunization. Hematoxylin-eosin-stained and toluidine blue-stained spinal cord sections from representative rats were examined for inflammatory cell infiltration and demyelination without knowledge of the group of origin.

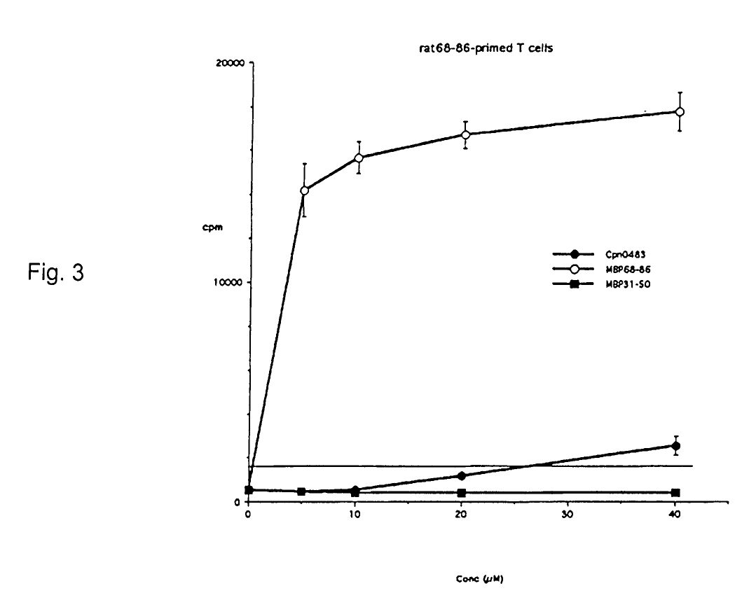

Initial clinical signs of EAE began to appear approximately 12 days post-immunization in groups given either dose, and those signs persisted for 3-6 days. The disease course from one experiment is shown in FIG. 2, and cumulative results are presented in Table 2. Clinical signs in most animals progressed from flaccid tail (grade 1) to complete hind-limb paralysis with incontinence (grade 3). At this time, most Cpn0483-immunized rats were sacrificed for studies of T cell proliferative responses; animals that were not sacrificed recovered from paralysis. As controls for the Cpn0483 peptide-immunized rats, additional groups of LEW rats (4 or 5/group) were immunized with 50 μg of rat or guinea pig MBP68-86 peptide, which induced clinical disease with comparable severity and time course to that induced by the C. pneumoniae-derived peptide (FIG. 2 and Table 2). Thus, the Cpn0483 peptide was encephalitogenic in this animal model. The lack of effect of the C>S substitution indicates that any effects of the presence of a Cys, possibly including disulfide bonding with other proteins, is not important for this activity.

TABLE 2
Induction of EAE in Lewis rats
with C. pneumoniae peptide Cpn0483.

| | EAE | EAE |
|---|---|---|
| Peptide | Incidence | Severity* |
| 5 μg Cpn0483 | 3/5 | 1.8 |
| 50 μg Cpn0483 | 14/15 | 2.3 |
| 5 μg Cpn0483 C > S | 4/5 | 2.4 |
| 50 μg Cpn0483 C > S | 4/5 | 2.2 |
| 50 μg rat68-86 | 10/10 | 2.7 |
| 50 μg gp68-86 | 5/5 | 3.0 |

*Mean group severity (max. = 3.0).

Extensive perivascular cuffing and parenchymal mononuclear cell infiltration was present in the spinal cords of the Cpn0483-immunized rats. Both are characteristic pathological findings in the rat model of EAE (Zamvil et al., supra; Swanborg, 1995, supra) and are also observed in MS (Raine et al., 1999, supra). Significant demyelination was not observed in Luxol fast blue-stained sections. This feature is prominent in MS, but is not characteristic of acute EAE in LEW rats. When spleen cells were prepared from Cpn0483-immunized LEW rats with EAE and activated in vitro with the same peptide for 72 hr, they transferred clinical disease to 6 of 9 syngeneic recipients. Six of 9 recipients of Cpn-primed spleen cells activated with MBP68-86 also developed clinical EAE. Mononuclear infiltration was present in the spinal cords of recipients with clinical disease, as expected.

### EXAMPLE III

Reactivity of T Cells from Rats Immunized with the Cpn0483 Peptide

To further investigate the immunopathology underlying the clinical observations, T cells were isolated from the spleens of rats immunized with Cpn0483 or MBP68-86, and recall responses were assessed using standard T cell proliferation assays.

Briefly, splenocytes were isolated from peptide-primed rats, adherent cells were removed by culture on plastic Petri dishes, and T cells were isolated on T cell columns (Biotec, Edmonton, Canada). The T cells were cultured for 96 hr with irradiated (2000rad) syngeneic thymocytes as antigen presenting cells (APCs), and peptide, in 96-well flat-bottom microtiter plates. The cultures were pulsed with ³H-thymidine (0.5 μCi/well) 18 hr prior to harvesting cells, and 3H-thymidine incorporation was measured in a liquid scintillation counter (Wallac 1450 Microbeta Plus, Gaithersburg, Md.). Cultures were run in quadruplicate and each experiment was repeated at least twice. Dose-response studies were performed using various peptides at differing concentrations, and representative results are presented.

T cells from rats immunized with the chlamydial peptide responded vigorously to the priming peptide. Moreover, they responded significantly to rat MBP68-86 (FIG. 3). In contrast, T cells derived from rats immunized with rat MBP68-86 proliferated vigorously to the priming peptide, but cross-reacted only minimally with the Cpn0483 homolog peptide at relatively high concentrations (FIG. 4). Immunological specificity was demonstrated by the lack of proliferation in response to an irrelevant nonencephalitogenic peptide (MBP11-30 or MBP31-50). T cells derived from unimmunized rats showed no proliferative response to any of the peptides tested.

The cross reactivity of Cpn0483-primed T cells with MBP68-86 probably reflects activation of self-MBP reactive T cells in the host. In contrast, one can speculate that the failure of MBP68-86-primed T cells to respond significantly to Cpn0483 may reflect the fact that the rats were not previously exposed to this exogenous microbe. Nevertheless, T cells from C. pneumoniae peptide-primed rats cross-react with MBP68-86, consistent with predictions of the molecular mimicry hypothesis.

A short-term T cell line from Cpn0483 peptide-immunized rats secreted IFN-γ (7,000 pg/ml) when activated for 72 hr with the chlamydial peptide, measured using commercial ELISA kits, but these cells did not produce detectable IL-4, confirming that the chlamydial peptide elicited an inflammatory Th1 response. It has been well established that EAE in rodents is mediated by IFN-γ-producing Th1 inflammatory cells ((Zamvil et al., supra; Swanborg, supra; R. B. Smeltz et al., J. Immunol. 162, 829 (1999)).

### EXAMPLE IV

Studies with Analogs of Cpn0483 and MBP68-86.

MBP68-86 and Cpn0483 share a YGxLxxxxxRTxDxN motif (SEQ ID NO: 17), see Table 3.

TABLE 3
Activity of Cpn0483 and RMBP D>A Analogue in Lewis rats

| | | | EAE | EAE |
|---|---|---|---|---|
| Peptide | SEQUENCE | | Incidence^{a} | Severity |
| MBP 68-86^{b} | YGSLPQKSQRTQDENPV | (SEQ ID NO:18) | 5/5 | 3.0 |
| MBP D>A | YGSLPQKSORTQAENPV | (SEQ ID NO:19) | 0/10 | |
| Cpn0483 | RFPNHYGCLLPRNPRTEDQN | (SEQ ID NO:3) | 5/5 | 3.0 |
| Cpn D>A | RFPNHYGCLLPRNPRTEAQN | (SEQ ID NO:4) | 9/10 | 2.7 |
| Motif | YGLxxxxxRTxDxN^{c} | (SEQ ID NO:17) | | |

^{a}Incidence at 50 μg (mean group severity max. = 3.0).
^{b}Native peptide sequence
^{c}x = any amino acid

The aspartic acid (D) residue is reportedly a T cell receptor contact for reactivity of guinea pig MBP73-86, the minimal encephalitogenic sequence, with LEW rat T cells (R. B. Smeltz et al., J. Immunol. 162, 829 (1999); M. U. M. Wauben et al., J. Exp. Med. 176: 667 (1992)).

To determine whether D is also required for Cpn0483-induced EAE in LEW rats, we prepared the Ala-substituted peptides Cpn0483 D>A, and rat MBP68-86-D>A and tested them for encephalitogenic activity in LEW rats. We confirmed earlier results that the replacement of D with A in MBP68-86 (MBP-D>A) abolished encephalitogenic activity for LEW rats (Smeltz et al., supra). In contrast, the A-substituted Cpn0483 analog (CpnD>A) elicited severe EAE in these animals (Table 3). These findings suggest that different specificity patterns, which presumably reflect activation of different subsets of encephalitogenic T cells, govern the induction of EAE by Cpn0483 and MBP68-86.

Other variants and truncated peptides of Cpn0483 and their demonstrated or predicted encephalitogenic activity are shown in Table IV, below (some results of which are also shown in the Tables above).

The above results show the importance of both the N-terminal and C-terminal amino acid residues of the 20 mer for encephalitogenic activity.

### EXAMPLE V

Induction of EAE with C. pneumoniae-Infected Hep-2 Cells.

To determine whether the C. pneumoniae 0483 protein could be processed and presented by LEW rat APCs to elicit evidence of EAE, we sonicated C. pneumoniae-infected Hep-2 cells and emulsified the sonicate in CFA. To minimize discomfort to the animals, the concentration of mycobacteria in the CFA was reduced to half the amount normally employed in encephalitogenic emulsions. Five rats were immunized with 0.05 ml of the emulsion containing 175 μg protein (total Hep-2- and C. pneumoniae-derived protein). Five control rats received emulsion containing 175 μg of protein from uninfected Hep-2 cells. One of the rats that received the C. pneumoniae emulsion exhibited limp tail consistent with EAE. Focal mononuclear cell infiltrates were present in the spinal cord of this rat. Neither the remaining four C. pneumoniae-immunized rats, nor the five control rats exhibited evidence of EAE. The low incidence of disease is not surprising, given that the sonicate contained the complete range of Hep-2 and C. pneumoniae proteins in relatively low overall dose. Thus, it is unlikely that Cpn0483 protein was present at optimal concentration to induce severe EAE. Furthermore, the CFA contained a suboptimal concentration of mycobacteria.

TABLE IV

| PEPTIDE | SEQ ID NO | SEQUENCE | EAE* |
|---|---|---|---|
| R-MBP-68-86 | 17 | Y G S L P Q K S Q R T Q D E N P V | 5/5 (3.0) |
| R>A | 18 | Y G S L P Q K S Q A T Q D E N P V | 6/8 (0.9) |
| N>A | 19 | Y G S L P Q K S Q R T Q D E A P V | 6/8 (1.6) |
| Cpn0483 | 3 | R F P N H Y G C L L P R N P R T E D Q N | 5/5 (3.0) |
| D>A | 4 | R F P N H Y G C L L P R N P R T E [symbol] Q N | 9/10 (2.7) |
| R>A | 5 | R F P N H Y G C L L P R N P [symbol] T E D Q N | 0/8 |
| R>N | 6 | R F P N H Y G C L L P R N P [symbol] T E D Q N | 0/4 |
| R>E | 7 | R F P N H Y G C L L P R N P [symbol] T E D Q N | 0/4** |
| N>A | 8 | R F P N H Y G C L L P R N P R T E D Q [symbol] | 1/8 (0.1) |
| N>R | 9 | R F P N H Y G C L L P R N P R T E D Q [symbol] | 0/4** |
| N>D | 10 | R F P N H Y G C L L P R N P R T E D Q [symbol] | 0/4** |
| C>S | 11 | R F P N H Y G S L L P R N P R T E D Q N | 8/10 (2.4) |
| N5—truncated | 12 | Y G C L L P R N P R T E D Q N | 0/5 |
| N2—truncated | 13 | P N H Y G C L L P R N P R T E D Q N | 0/5 |
| C5—truncated | 14 | R F P N H Y G C L L P R N P R | 0/5 |
| N3/C2 truncated | 15 | N H Y G C L L P R N P R T E D | 0/5 |
| N4/C2 truncated | 16 | H Y G C L L P R N P R T E D | 0/5 |
| Motif | | Y G x L x x x x x R T x D x N | |

*Incidence (# affected animals/total # animals inejected); severity score in parentheses as above
**Predicted results

### DISCUSSION OF EXAMPLES

The results presented here demonstrate that a 20-mer amino acid sequence intrinsic to a Chlamydia pneumoniae-specific protein of unknown function elicits MS-like clinical disease, and MS-like aspects of spinal cord pathology, in the LEW rat. Only 6 amino acids in the Cpn0483 peptide are identical to the cognate sequence in rat MBP68-86 (Tables 1 and 3). Studies in mice (A. M. Gautam et al., J. Exp. Med. 176, 605, (1992)) and DA rats (R. B. Smeltz et al., J Neuroimmunol. 87, 43, 1998) revealed that poly-Ala peptides with five or six native residues in the correct MBP configuration are encephalitogenic. These residues provide a structural motif that permits interaction of the peptide with major histocompatibility complex (MHC) class II gene products. This peptide-MHC complex, in turn, interacts with specific T cell receptors (TCR) and initiates T cell activation. Although some particular amino acids at specific positions in disease-eliciting peptides have been shown to be critical for disease induction, presumably because they interact directly with the TCR (Smeltz et al., 1999, supra), it has also been demonstrated that TCRs can recognize different but structurally related peptides (K. W. Wucherpfennig et al., Cell 80, 695 (1995); B. Hemmer et al., J. Immunol. 160, 3631 (1998)).

The 20 amino acid chlamydial peptide appears to be as effective as guinea pig or rat MBP68-86 peptide in causing paralysis in this model. It is not yet clear which of the common MBP and Cpn0483 residues are critical for disease induction.

C. pneumoniae has been shown to be a highly unusual pathogen over the decade since its identification. During that time the organism has been associated not only with respiratory disease but also with chronic obstructive pulmonary disease, atherosclerosis, temporal arteritis, MS, and late-onset Alzheimer's and other diseases (e.g., Sriram et al., supra; B. J. Balin et al., Med Microbiol Immunol 187, 23 (1998); J. B. Muhlestein et al., J. Am. Coll. Cardiol. 27, 1555 (1996); A. D. Wagner et al., Arthritis Rheum. 43, 1543 (2000)). While the role of C. pneumoniae in MS remains controversial (M. R. Hammerschlag et al., J. Clin. Microbiol. 38:4274 (2000)), the results presented here appear consistent with an infectious etiology for this disease in at least a subset of patients. A number of investigators have postulated, and presented results supporting, such an infectious etiology. It would be significant to try to relate the epidemiology of C. pneumoniae to the incidence and prevalence of MS in areas where an infectious causation has been postulated, e.g., the Faroe Islands (J. F. Kurtzke, J. Neurovirol. 6: Suppl. 2, S134 (2000)).

Even if this microbe is involved in MS, consideration will have to be given to direct vs. indirect effects. In this regard, it has recently been reported that the APO ε4 allele is associated with faster progression to disability in MS (J. Chapman et al., Neurology 56: 312 (2001). Importantly, a recent study showed that 68% of patients with C. pneumoniae-associated arthritis possess the APOε4 allele (H. C. Gerard et al., Microbial Pathogenesis 26: 35 (1999)). Thus, exposure of individuals expressing certain genes (e.g., the APO ε4 allele) to the appropriate infectious agent (e.g., C. pneumoniae) may play a role in the induction of MS.

Regardless, there are clear differences between EAE in rats and MS. The former is an acute inflammatory disease with scant demyelination, whereas demyelination is a prominent feature of MS. MBP reactive T cells in MS patients are predominantly directed toward a sequence contained within residues 84-102, whereas the dominant encephalitogenic epitope for LEW rats is comprised of MBP68-86. The 84-102 peptide contains the sequence KNIVTPRTPPP [SEQ ID NO: 26], and a Blast search by the present inventors turned up a chlamydial gene, Cpn0442, specifying a protein containing the sequence KNLFPPYEPPP (SEQ ID NO: 27), which could activate human MBP-reactive T cells. In support of this contention is the report that human papillomavirus 7 contains a VHFFK (SEQW ID NO: 28) motif identical to a sequence also present in MBP87-99 (R. L. Ufret-Vincenty et al., J. Exp. Med. 188:1725 (1998)). The viral peptide is capable of selecting papillomavirus-specific SJL mouse T cells that cross-react with MBP87-99, a major encephalitogenic epitope for SJL mice. The papillomavirus-specific T cells proliferate to both the viral and MBP peptides, and are encephalitogenic for SJL mice (Ufret-Vincenty et al., supra).

The present Examples reveal that a C. pneumoniae-derived peptide is capable of inducing autoimmune central nervous system disease in a rodent model of the disease.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A composition comprising a Chlamydia pneumoniae (Cpn) protein 0483 (SEQ ID NO: 2) or a peptide fragment thereof or functional derivative of the peptide fragment, that stimulates Th1 cells that are specific for a myelin basic protein (MBP) autoantigen and, which, when administered to Lewis rats, induces severe experimental autoimmune encephalomyelitis in a majority of animals.

2. The composition of claim 1 encoded by all or part of the DNA molecule having SEQ ID NO: 1.

3. The composition of claim 1 or 2 wherein the peptide includes the motif
YGxLxxxxxRTxDxN (SEQ ID NO: 17),
wherein x is any amino acid.

4. The composition of any of claims 1-3 wherein the peptide fragment has the sequence SEQ ID NO: 3.

5. The composition of any of claims 1-4 that is the peptide RFPNHYGCLLPRNPRTEDQN (SEQ ID NO: 3) or is a Th1-stimulatory and/or encephalitogenic functional derivative thereof.

6. The composition of claim 5 wherein the functional derivative is RFPNHYGCLLPRNPRTEAQN (SEQ ID NO: 4) or RFPNHYGSLLPRNPRTEDQN (SEQ ID NO: 11).

7. A composition that is a non-encephalitogenic polypeptide comprising SEQ ID NO: 3, or that comprises a peptide analogue or functional derivative of SEQ ID NO: 3.

8. The composition of claim 7 selected from the group consisting of:

| | | |
|---|---|---|
| (a) | RFPNHYGCLLPRNPATEDQN; | (SEQ ID NO:5) |
| (b) | RFPNHYGCLLPRNPNTEDQN; | (SEQ ID NO:6) |
| (c) | RFPNHYGCLLPRNPETEDQN; | (SEQ ID NO:7) |
| (d) | RFPNHYGCLLPRNPRTEDQA; | (SEQ ID NO:8) |
| (e) | RFPNHYGCLLPRNTPRTEDQR; | (SEQ ID NO:9) |
| (f) | RFPNHYGCLLPRNPRTEDQD; | (SEQ ID NO:10) |
| (g) | YGCLLPRNPRTEDQN; | (SEQ ID NO:12) |
| (h) | PNHYGCLLPRNPRTEDQN; | (SEQ ID NO:13) |
| (i) | RFPNHYGCLLPRNPR; | (SEQ ID NO:14) |
| (j) | NHYGCLLPRNPRTED; and | (SEQ ID NO:15) |
| (k) | HYGCLLPRNPRTED. | (SEQ ID NO:16) |

9. The composition of claim 7 wherein the polypeptide has between about 20 and 50 amino acids.

10. A complex between and MHC class II protein and a peptide which complex is capable of inducing unresponsiveness or less responsiveness in a T cell that is specifically immunoreactive with an autoantigen and which T cell is an effector cell or regulatory cell in the pathogenesis of MS, the complex comprising
(a) a peptide that includes SEQ ID NO: 3 or includes a functional derivative thereof covalently bound to
(b) an isolated MHC class II component having an antigen binding pocket,
wherein the antigenic peptide is physically associated with the antigen binding pocket and is recognized by the T cell receptor of the reactive T cell.

11. The complex of claim 10, wherein the peptide is covalently bound via a peptide linkage to an MHC class II chain and is non-covalently associated with the antigen binding pocket.

12. The complex of claim 10 or 11 wherein the peptide comprises an epitope recognized by a T cell receptor of a T cell specifically immunoreactive with an MBP autoantigen.

13. The complex of any of claims 10-12 wherein the peptide is a non-encephalitogenic peptide analogue or functional derivative of SEQ ID NO: 3.

14. The complex of claim 13 wherein the peptide analogue is selected from the group consisting of

| | | |
|---|---|---|
| (a) | RFPNHYGCLLPRNPATEDQN; | (SEQ ID NO:5) |
| (b) | RFPNHYGCLLPRNPNTEDQN; | (SEQ ID NO:6) |
| (c) | RFPNHYGCLLPRNPETEDQN; | (SEQ ID NO:7) |
| (d) | RFPNHYGCLLPRNPRTEDQA; | (SEQ ID NO:8) |
| (e) | RFPNHYGCLLPRNPRTEDQR; | (SEQ ID NO:9) |
| (f) | RFPNHYGCLLPRNPRTEDQD; | (SEQ ID NO:10) |
| (g) | YGCLLPRNPRTEDQN; | (SEQ ID NO:12) |
| (h) | PNHYGCLLPRNPRTEDQN; | (SEQ ID NO:13) |
| (i) | RFPNHYGCLLPRNPR; | (SEQ ID NO:14) |
| (j) | NIHYGCLLPRNIPRTED; and | (SEQ ID NO:15) |
| (k) | HYGCLLPRNPRTED. | (SEQ ID NO:16) |

15. A pharmaceutical composition comprising;
(a) the composition of any of claims 1-6 or a pharmaceutically acceptable salt thereof; and
(b) a pharmaceutically acceptable carrier or excipient.

16. A pharmaceutical composition comprising;
(a) the composition of any of claims 7-9 or a pharmaceutically acceptable salt thereof; and
(b) a pharmaceutically acceptable carrier or excipient.

17. A pharmaceutical composition comprising;
(a) the complex of any of claims 10-14 or a pharmaceutically acceptable salt thereof; and
(b) a pharmaceutically acceptable carrier or excipient.

18. A method for inhibiting a Th1 lymphocyte response to a peptide or protein that includes the peptide motif YGxLxxxxxRTxDxN (SEQ ID NO: 17), wherein x is any amino acid, or that is induced by Cpn0483 peptide SEQ ID NO: 3, which method comprises providing to a population of lymphocytes that includes Th1 cells an effective amount of the composition of any of claims 7-9 to inhibit the Th1 response.

19. A method for inhibiting a Th1 lymphocyte response to a peptide or protein that includes the peptide motif YGxLxxxxxRTxDxN (SEQ ID NO: 17), wherein x is any amino acid, or that is induced by Cpn0483 peptide SEQ ID NO: 3, which method comprises providing to a population of lymphocytes that includes Th1 cells an effective amount of the complex of claim 13 or 14 to inhibit the Th1 response.

20. The method of claim 18 or 19 wherein the providing is in vivo.

21. A method for inducing a Th2 immune response to an autoantigenic peptide associated with MS in a subject, comprising contacting the immune system of the subject with a composition of any of claims 7-9, optionally in combination with a cytokine or other agent that promotes activation of Th2 lymphocytes.

22. A method for inducing a Th2 immune response to an autoantigenic peptide associated with MS in a subject, comprising contacting the immune system of the subject with a complex of claim 13 or 14, optionally in combination with a cytokine or other agent that promotes activation of Th2 lymphocytes.

23. The method of claim 21 or 22 wherein the cytokine is IL-4.

24. The method of any of claim 21-23 wherein the contacting is in vivo.

25. A method of treating a subject having, or at risk for, multiple sclerosis and in need of treatment or prophylaxis therefor, comprising administering to the subject an effective amount of the pharmaceutical composition of claim 16 or 17.

26. A genetically modified mammalian cell comprising a polynucleotide encoding a Cpn polypeptide 0483 (SEQ ID NO: 2), a Cpn peptide SEQ ID NO: 3, or a functional derivative of the peptide.

27. A genetically modified mammalian cell comprising a polynucleotide having SEQ ID NO: 1 or a fragment thereof which polynucleotide or fragment is expressed in or on the cell, the polypeptide or peptide product of which stimulates Th1 cells that are specific for a MBP auto antigen.

28. A genetically modified mammalian cell comprising a polynucleotide encoding a non-encephalitogenic
(a) polypeptide comprising SEQ ID NO: 3, or
(b) peptide analogue or functional derivative of SEQ ID NO: 3.

29. A population of cells that were transfected or transduced with an exogenous polynucleotide that encodes the peptide or functional derivative of any of claims 7-9, wherein the cells express the polypeptide, peptide or functional derivative when they are administered to a subject in vivo, and wherein, when the subject has multiple sclerosis, the presence of the polypeptide, peptide or functional derivative delays onset, prevents or diminishes the progression or severity or the multiple sclerosis.

30. The cell of claim 26 or 27 that is a human cell.

31. The cell or cells of claim 28 or 29 that are human cells.

32. A method of treating a subject having, or at risk for, multiple sclerosis and in need of treatment or prophylaxis therefor, comprising administering to the subject an effective amount of cells according to claim 28, 29 or 31 that are autologous or otherwise compatible with the subject, thereby treating the subject.

32. A method of treating a subject having, or at risk for, multiple sclerosis and in need of treatment or prophylaxis therefor, comprising the steps of:
(a) obtaining T cells from the subject,
(b) optionally, enriching Th2 cells from the T cells;
(d) expanding T cells of step (a) or (b) in culture in the presence of the compositions of any of claim 7-9, optionally in the presence of growth factors or accessory or feeder cells; to obtain protective T cells
(e) administering to the subject an effective amount of the protective T cells, thereby providing the treatment or prophylaxis to the subject.

33. The method of claim 32 further comprising administering to the subject
(a) an agent with promotes the survival or action of the cells, and/or
(b) a drug that treats any symptom of multiple sclerosis.

34. A conjugate consisting of the polypeptide, peptide or functional derivative of any of claims 1-9 conjugated to a second molecule.

35. A conjugate consisting of the complex of any of claims 10-14 conjugated to a second molecule.

36. The conjugate of claim 34 or 35 in which the second molecule is a detectable label.

37. The conjugate of claim 34 or 35 in which the second molecule is a polypeptide.

38. The conjugate of claim 34 or 35 in which the second molecule is a small organic molecule.


SEQUENCE LISTING (28 sequences)

SEQ ID NO: 1 - DNA, 3132 bp, Chlamydia pneumoniae
  CDS (1)..(3129)
ttgattaaaaaacgagcaatttttgaacgtatgtttccaattcccccaccacattgcccgcccaataacaagaataatttttaccacttaacgactgatactaaagaccctctgttacttagaattctacgtaccataggatacgttctgctccatatcattactcttggtttgcttcttctgattcactactacaagcatcatcgggttgtcagaaaagaaggcttgccaacgcctcccactcttcccaaaggaccagagccaaaaactatagaaattgccaaacaaccgcctaaggatggtgaagacaaaaaacccgatgttcccaagccgggcacgccgcccccagaggacacacccccgcctccccccaaagctccttcaccagcgagcccaaaagtccctaaacaacctgctgataaaaagccgactccaccaccagaggcccctcctcctcccgtacgggtggctacccccatgcctctccgcccatctagtcaaggctattggcaatgcttaaatcgcatggtgagcatggtactaagacgagcgcctctgcctcttcctgccatgcaagttgatccaatacttggcgactttaaccctcatttcgtagcttcctatcccaatcggattgataacgaaccgatgtatttccaaataaaacagttcaagaaaatcgcacaaaatccggatcttcctcaacaacaccggcgacttgcgcaactctctcttgaacaggctctctatctaaatgacaattactaccttgtgaatgtaccgggagatgggaactgcttttatcgtgcctatgctgtaggatggctatctgctctctacgaagagagcagcagaaatgatattgtctttgagcaggaagccacacgtctccttgacctgcctttcgcctcctcttctccggcaaatgcgaatctttgtgcagaaatggctgaactccttcagttatgcagtacttattgctccttcatagacctctatgacggggtgattctttctcagaaacacactgcaactctgatagcctttctaagaaaactctctgcatatgcgattcgccaacaaatcgcagcttcaagtaatgaagaaacagcgagagccttatttatttctgatatgcaggacgatctcctccccagtgttctggaatttcttgctgcaaatcgtccctattcggaattgttccaaaatctcattgatcattccgcacttccttacatgcaatctagagacaaactctttcttctcttggaacatctgcccgctctctttcttactgatgcagagcttcaaaagatgtctccagaagatcaacaacttcgaaagcaatatgaaagagaaatacgagaggcttttgctaagctgagtcgacgcattgctgattcagggtgggatactgagagattcaatgctatagtcaaagatcacctccctgaagcaatccgatgtcaatactctcgctttcttgcaactatagaaaacagacgatctggggatctcccttggtctccagctctttctttctttgcttttctatgtacctgcccctctgtaagatttcacaaactctgcgctactttctacaaatcattagaggatatcattatagcgtccgcgcccccccaacgctctatacaagagatcttacaaataagtaacgcctccctcagctaccttaatgaagatttagattcttcttggcaacgagaggtgatttcttctaacatcatgactatccttacgactcatgagagtttgacgttagagagctctatgcctcaactcgaaacactacataaacgcatagcaaacctattaaagaatgtaatatccacatcctttgaaacccctcctttaagcaatcagccggatttactttcaaatcttgtaaacaagctattagtcgcaattcatagtaagcttgaattaaaagagcacttcaatactgtctgctcggcaagaagtttacgtttaacgcgtgatgaaggcagtggtctctcacaagagcaggacctcctctatacacaggcagtacagctcttattctttattttacagcatcctcaagtgaataatcgtccagaaactaaagatgccgttaaagagttaaaaatgcttctacttccttttctacaatatgcctttaaaaaagtagaaaacgaaaagaaactccaaaaacttctacgttccattctagggtctctagtactcaagcctccagcacgctatccttcaaccccttctaataaagataaagagacgttctgcaagttctggtcacgacatcctgaagtgatggttttagatcccatacttgaaaagaactgtatgcagtttctacgagctactttcccaaattatcaactggaaaccgaggccatactcttagaaaaagaaatcgaaagtacctttaggaatgggtggaacgtttttttaacacggttaaatctcttcggatcaaaactgggttcgccttcttctcccacagctttaagtgatcagttttcgaaatcttttttaatcttttgtttccttaacaactaccctaaacttctacaaaaaaagactccgctagctgctcgattagacgctttccaaagagaggcttctcatagatttacacaagtaaaagataagcttttactttcgttaaaatacggtttccctctagctacagcgactataaatcaatactctagagctcgagatcagttgatttgtaatctcttaaaaaacacggtcacagcatctgatggtttctgtcgctctggttttagacaatcactgataggctacctccactccctaagttctaatgaactcggtgatatcttggatgacgtcaaagagcaagctgaggctaacgacgtcgctgctatgactactgtacctttgcagccgtttgctgtttgtctgatcatgtctgatcgagatactgtctcagaagaaaatattgaaaactttgttgcgatgcatggatttttaaatacaatttctccggaaagagacgctcgtatcttcttaatccgcttccccaaccactacggttgtctcttgcctagaaaccctagaactgaagatcagaactcaaaaccggacagctcaaatccctag

SEQ ID NO: 2 - protein, 1043 aa, Chlamydia pneumoniae
LIKKRAIFERMFPIPPPHCPPNNKNNFYHLTTDTKDPLLLRILRTIGYVLLHIITLGLLLLIHYYKHHRVVRKEGLPTPPTLPKGPEPKTIEIAKQPPKDGEDKKPDVPKPGTPPPEDTPPPPPKAPSPASPKVPKQPADKKPTPPPEAPPPPVRVATPMPLRPSSQGYWQCLNRMVSMVLRRAPLPLPAMQVDPILGDFNPHFVASYPNRIDNEPMYFQIKQFKKIAQNPDLPQQHRRLAQLSLEQALYLNDNYYLVNVPGDGNCFYRAYAVGWLSALYEESSRNDIVFEQEATRLLDLPFASSSPANANLCAEMAELLQLCSTYCSFIDLYDGVILSQKHTATLIAFLRKLSAYAIRQQIAASSNEETARALFISDMQDDLLPSVLEFLAANRPYSELFQNLIDHSALPYMQSRDKLFLLLEHLPALFLTDAELQKMSPEDQQLRKQYEREIREAFAKLSRRIADSGWDTERFNAIVKDHLPEAIRCQYSRFLATIENRRSGDLPWSPALSFFAFLCTCPSVRFHKLCATFYKSLEDIIIASAPPQRSIQEILQISNASLSYLNEDLDSSWQREVISSNIMTILTTHESLTLESSMPQLETLHKRIANLLKNVISTSFETPPLSNQPDLLSNLVNKLLVAIHSKLELKEHFNTVCSARSLRLTRDEGSGLSQEQDLLYTQAVQLLFFILQHPQVNNRPETKDAVKELKMLLLPFLQYAFKKVENEKKLQKLLRSILGSLVLKPPARYPSTPSNKDKETFCKFWSRHPEVMVLDPILEKNCMQFLRATFPNYQLETEAILLEKEIESTFRNGWNVFLTRLNLFGSKLGSPSSPTALSDQFSKSFLIFCFLNNYPKLLQKKTPLAARLDAFQREASHRFTQVKDKLLLSLKYGFPLATATINQYSRARDQLICNLLKNTVTASDGFCRSGFRQSLIGYLHSLSSNELGDILDDVKEQAEANDVAAMTTVPLQPFAVCLIMSDRDTVSEENIENFVAMHGFLNTISPERDARIFLIRFPNHYGCLLPRNPRTEDQNSKPDSSNP

SEQ ID NO: 3 - protein, 20 aa, Chlamydia pneumoniae
RFPNHYGCLLPRNPRTEDQN

SEQ ID NO: 4 - protein, 20 aa, Chlamydia pneumoniae
RFPNHYGCLLPRNPRTEAQN

SEQ ID NO: 5 - protein, 20 aa, Chlamydia pneumoniae
RFPNHYGCLLPRNPATEDQN

SEQ ID NO: 6 - protein, 20 aa, Chlamydia pneumoniae
RFPNHYGCLLPRNPNTEDQN

SEQ ID NO: 7 - protein, 20 aa, Chlamydia pneumoniae
RFPNHYGCLLPRNPETEDQN

SEQ ID NO: 8 - protein, 20 aa, Chlamydia pneumoniae
RFPNHYGCLLPRNPRTEDQA

SEQ ID NO: 9 - protein, 20 aa, Chlamydia pneumoniae
RFPNHYGCLLPRNPRTEDQR

SEQ ID NO: 10 - protein, 20 aa, Chlamydia pneumoniae
RFPNHYGCLLPRNPRTEDQD

SEQ ID NO: 11 - protein, 20 aa, Chlamydia pneumoniae
RFPNHYGSLLPRNPRTEDQN

SEQ ID NO: 12 - protein, 15 aa, Chlamydia pneumoniae
YGCLLPRNPRTEDQN

SEQ ID NO: 13 - protein, 18 aa, Chlamydia pneumoniae
PNHYGCLLPRNPRTEDQN

SEQ ID NO: 14 - protein, 15 aa, Chlamydia pneumoniae
RFPNHYGCLLPRNPR

SEQ ID NO: 15 - protein, 15 aa, Chlamydia pneumoniae
NHYGCLLPRNPRTED

SEQ ID NO: 16 - protein, 14 aa, Chlamydia pneumoniae
HYGCLLPRNPRTED

SEQ ID NO: 17 - protein, 15 aa, Unknown
  Consensus sequence
YGXLXXXXXRTXDXN

SEQ ID NO: 18 - protein, 17 aa, Rattus sp.
YGSLPQKSQRTQDENPV

SEQ ID NO: 19 - protein, 17 aa, Rattus sp.
YGSLPQKSQRSQAENPV

SEQ ID NO: 20 - protein, 17 aa, Rattus sp.
YGSLPQKSQRSQDEAPV

SEQ ID NO: 21 - protein, 17 aa, Guinea pig
YGSLPQKSQRSQDENPV

SEQ ID NO: 22 - DNA, 28 bp, Unknown
  Primer
ctgtatgcagtttctacgagctactttc

SEQ ID NO: 23 - DNA, 25 bp, Unknown
  Primer
cggattaagaagatacgagcgtctc

SEQ ID NO: 24 - DNA, 27 bp, Unknown
  Primer
aagactccgctagctgctcgattagac

SEQ ID NO: 25 - DNA, 25 bp, Unknown
  Primer
gtcatagcagcgacgtcgttagcct

SEQ ID NO: 26 - protein, 11 aa, Rattus sp.
KNIVTPRTPPP

SEQ ID NO: 27 - protein, 11 aa, Chlamydia pneumoniae
KNLFPPYEPPP

SEQ ID NO: 28 - protein, 5 aa, Rattus sp.
VHFFK